SEQUENCE LISTING (10 sequences)

SEQ ID NO: 1 - DNA, 47 bp, Artificial Sequence
  targeting strand mdx-r6
taguugaagccauuuuauugcucuuucaaagaacgcgcguuuucgcg

SEQ ID NO: 2 - DNA, 50 bp, Artificial Sequence
  mutator strand mdx-6-1
gttctttgaaagagcaacaaaatggcttcaactatctgagttttctcaga

SEQ ID NO: 3 - DNA, 50 bp, Artificial Sequence
  mutator strand mdx-6-2
gttctttgaaagagcagcaaaatggcttcaactatctgagttttctcaga

SEQ ID NO: 4 - DNA, 42 bp, Artificial Sequence
  targeting strand A
guugugcccagtcctagccgaauagcctctccttttggagag

SEQ ID NO: 5 - DNA, 41 bp, Artificial Sequence
  mutator strand B
gctattcggctacgactgggcacaacagacaattttttgtc

SEQ ID NO: 6 - DNA, 70 bp, Artificial Sequence
  Primary Sequence of Duplex Mutational Vector S
gctattcggctacgactgggcacaattttuugugcccagtcctagccgaauagcctctccttttggagag

SEQ ID NO: 7 - DNA, 44 bp, Artificial Sequence
  Exon 7 bHMV mutator strand
atgtgtgtgtttcaggccagacctgtttgattgguuuuccaatc

SEQ ID NO: 8 - DNA, 44 bp, Artificial Sequence
  Exon 7 bHMV targeting strand
aaacaggucuggcccgaaacacacacaugcgcgcuuuugcgcgc

SEQ ID NO: 9 - DNA, 49 bp, Artificial Sequence
  Exon 8 bHMV mutator strand
ggataagtggtggcaaatctgtaagcacattaacaccaguuuuctggtg

SEQ ID NO: 10 - DNA, 54 bp, Artificial Sequence
  Exon 8 bHMV targeting strand
tuaaugugcuuacagauguugccaccacuuauccgcgcgcgcuuuugcgcgcgc

(19) United States
(12) Patent Application Publication
(10) Pub. No.: US 2003/0017593 A1
(43) Pub. Date: Jan. 23, 2003

(54) Binary hybrid mutational vectors

(76) Inventors: Howard B. Gamper, Philadelphia, PA (US); Eric B. Kmiec, Landenburg, PA (US); Richard J. Bartlett, Weston, FL (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 09/874,720

(22) Filed: Jun. 5, 2001

Related U.S. Application Data
(60) Provisional application No. 60/209,530, filed on Jun. 5, 2000.

(51) Int. Cl. C12N 15/87

(52) U.S. Cl. 435/455; 435/320.1

(57) ABSTRACT

The invention concerns novel compounds used to make specific genetic alterations in the genome of target eukaryotic and prokaryotic cells, methods of their production and use, and kits containing the compounds. The compounds are two-strand duplex oligonucleobase compounds termed binary Hybrid Mutational Vectors (bHMV). A bHMV preferably contains a deoxyribo-type oligonucleobase mutator strand and a ribo-type oligonucleobase targeting strand. A bHMV is constructed to effect changes in a target gene via a combination of gene targeting and mismatch repair between the bHMV and the target gene.

The benefit of the filing date of copending U.S. Provisional Application Serial No. 60/209,530 filed Jun. 5, 2000 is hereby claimed.

### FIELD OF THE INVENTION

The invention relates to oligonucleobase compounds used to make specific genetic alterations in the genome of target eukaryotic and prokaryotic cells.

### BACKGROUND OF THE INVENTION

Those skilled in the art of molecular biology recognize a need to introduce specific alterations into the genetic sequence of a eukaryotic or prokaryotic genome. For example, a mutation can be created in a specific gene of a eukaryotic or prokaryotic cell for the purposes of studying gene function. Alternatively, an existing mutation can be repaired to correct a detrimental genetic defect. Cells carrying a specific genetic alteration can be used to construct transgenic organisms. The specific alteration of genetic sequences is also useful in gene therapy. Of particular interest are gene therapy techniques involving the removal, repair and replacement of hematopoietic stem cells in individuals affected with certain hemoglobinopathies (see U.S. Pat. Nos. 5,760,012 and 5,888,983 of Kmiec et al.).

The process of introducing genetic material into cultured eukaryotic cells is called “transfection.” A variety of transfection techniques have been developed, including calcium phosphate precipitation, DEAE-dextran mediated endocytosis, electroporation, and liposome mediated fusion. However, performing these transfection techniques without specialized transfection or mutational vectors results in the random insertion of genetic material into the genome. The random insertion of genetic material into the genome is called “illegitimate recombination.” There is thus a need for compounds and methods of their use that avoid illegitimate recombination and introduce alterations at specific sites on target gene.

Some site-specific alteration of a target gene was attained in higher eukaryotic cells through the use of very long (>1 kb) nucleic acids. However, elaborate selection schemes were needed to isolate the “true” recombinants, because the rate of illegitimate recombination still far exceeded the rate of homologous recombination in these systems. Thomas, K. R. and Capecchi, M. R., 1987, Cell 52:503. See also Valancius, V. and Smithies, O. 1991, Mol. Cell. Biol. 11:4389 (comparison of homologous recombination of linearized and supercoiled plasmids in eukaryotic cells).

A predominantly site-directed mutagenesis was achieved in the yeast Saccharomyces cerevisiae by the direct injection of single-stranded oligodeoxynucleotides. However, S. cerevisiae has a higher natural rate of homologous recombination than the higher eukaryotes, Moerschell, R.P. et al. 1988. Proc. Natl. Acad. Sci. 85:524-28; Yamamoto, T. et al. 1992. Yeast 8:935-48, and to date this technique has proven ineffective for transfecting mammalian and avian cells.

U.S. Pat. Nos. 5,731,181, 5,795,972, 5,565,350, 5,756,325, and 5,871,984 of Kmiec describe a duplex mutational vector capable of specifically altering a target gene sequence. Kmiec called this vector a “Chimeric Mutational Vector” or “CMV” because it consists of mixed ribo-deoxyribo oligonucleobase strands designed to target and alter a specific gene sequence. The CMV has duplex regions that are homologous to the target gene, and a duplex mutator region.

When RNA is present in both strands, the mixed ribo-deoxyribo oligonucleobase strands of the CMV can reduce the CMV's efficiency for introducing genetic alterations. Also, because different synthetic schemes must be employed to make the mixed ribo-deoxyribo oligonucleobase strands, the CMV is difficult and costly to synthesize and purify.

Furthermore, assembly of a CMV is accomplished by the cumbersome ligation of multiple-molecule complexes. For example, one procedure describes the ligation of three separate molecules in a ternary complex (a sticky ended DNA duplex and two sticky ended chimeric hairpins). Another CMV assembly technique describes the ligation of four separate molecules in a quaternary complex (two separate oligonucleotide chains and two separate hairpin structures).

Kmiec also describes a circular CMV assembled through enzymatic ligation of a cohesive ends. The cohesive ends are formed by endonuclease digestion of artificial restriction enzyme sites located within the targeting region. However, artificial creation of restriction sites in the targeting region may introduce deleterious and/or unwanted alterations into the target gene sequence.

Kren et al., (1997) Hepatology 25, 1462-1468 describes single chain chimeric mutational vectors with a single base mismatch in the DNA portion of the molecule. U.S. Pat. No. 6,010,907 of Kmiec et al. discloses both chimeric and non-chimeric duplex mutational vectors that are a single oligonucleobase chain. Efficiency of transduction with such single-nicked vectors is reduced, likely due to conformational constraints on the vectors during recombination.

Thus, there is a need for mutational vectors, and methods of their use, wherein the mutational vectors are capable of effecting specific alterations in a target gene sequence without undergoing illegitimate recombination. There is also a need for mutational vectors which do not contain sequences that may introduce deleterious and/or unwanted alterations into the target gene sequence. There is a further need for mutational vectors wherein the structure and composition of the vectors allows for an efficient use of the cell's DNA recombination machinery.

### BRIEF DESCRIPTION OF THE FIGURES.

FIG. 1A is a schematic representation of an exemplary targeting strand according to the present invention.

FIG. 1B is a schematic representation of an exemplary mutator strand according to the present invention.

FIG. 2 is a schematic representation of a binary hybrid mutational vector (“bHMV”) according to the present invention. Positions A and B show the location of strand nicks.

FIG. 3 shows the formation of two different bHMV's (molecules C and F) according to the present invention. The first bHMV (molecule C) has two nicks on one side of the duplex (called the “staple” configuration), and is formed by hybridization of a targeting strand containing no hairpin structures (molecule A) to a mutator strand containing a hairpin structure at the 3′ and 5′ ends of the oligonucleobase chain (molecule B). The second bHMV (molecule F) has one nick on each side of the duplex (called the “double-hook” configuration), and is formed by the hybridization of a targeting strand containing a hairpin structure at the 3′ end of the oligonucleobase chain (molecule D) with a mutator strand containing a hairpin structure at the 3′ end of the oligonucleobase chain (molecule E).

FIG. 4 shows the construction of two bHMVs containing a common targeting strand and two different mutator strands in the “double hook” configuration. Molecule A has the primary sequence and secondary structure of targeting strand mdx-r6 (SEQ ID NO:1). Molecules B and D have the primary sequence and secondary structure of two mutator strands; mdx-6-1 (one base mismatch to targeting strand mdx-r6; SEQ ID NO:2) and mdx-6-2 (two base mismatch; SEQ ID NO:3). Molecules C and E comprise the bHMVs produced by the hybridization of mutator strands mdx-6-1 and mdx-6-2 to targeting strand mdx-r6, respectively. 2′-O-methyl ribonucleotides are lower case; deoxyribonucleotides are uppercase.

In FIG. 4, the primary sequences of molecules A, B and D are as follows (2′-O-methyl ribonucleotides in lowercase; deoxyribonucleotides in uppercase):

molecule A (targeting strand mdx-r6):

3′-gcgcuuuugcgcgcaagaaacuuucucguuauuuuaccgaaguugaT-5′ (SEQ ID NO:1)

molecule B (mutator strand mdx-6-1):

| | |
|---|---|
| 3′-AGACTCTTTTGAGTCTATCAACTTCGGTAAAACAACGAGAAAG | (SEQ ID NO:2) |
| TTTCTTG-5′ | |

molecule D (mutator strand mdx-6-2):

| | |
|---|---|
| 3′-AGACTCTTTTGAGTCTATCAACTTCGGTAAAACGACGAGAAAGTT | (SEQ ID NO:3) |
| TCTTG-5′ | |

FIG. 5A shows bHMV A/B as described in Example 1. bHMV A/B has two nicks, each nick being indicated by a propylamine group attached to the oligonucleobase strand.

FIG. 5B shows duplex mutational vector S as described in Example 2. Duplex mutational vector S has one nick, as indicated in the figure.

### DEFINITIONS

An “oligonucleobase strand” is a polymer of nucleobases having a 3′ and a 5′ end that can hybridize to complementary oligonucleobase sequences.

“Hybridization” is the joining of complementary oligonucleobases by Watson-Crick base-pairing to form a duplex. Hybridization can occur between oligonucleobases of the same strand, or between oligonucleobases of different strands. It is understood that under some conditions of low salt and/or elevated temperature the Watson-Crick base pairs may cease to be thermodynamically stable. It is also understood that a one or two base pair mismatch does not affect the ability of oligonucleobases to hybridize.

A “syngamic eukaryotic organism” is a eukaryotic organism that undergoes sexual reproduction by the union of gamete cells. As used herein, a “gamete” is any haploid cell (including a polar body) that is capable of fusing with, or can be caused to fuse with, another cell to form a zygote. As used herein, a “zygote” is a cell having the potential for development into a complete organism. Zygotes are typically diploid. A zygote can result from parthenogenesis or nuclear transplantation as well as the natural or artificial merger of two gametes.

A “mutation associated with a genetic disease” is any mutation whose presence in a genome of a subject is causative or correlative of the disease state in the subject.

A “nick” is the lack of a covalent bond between the sugar moieties of two adjacent nucleobases in a duplex molecule comprising oligonucleobases.

An oligonucleobase “consisting essentially of” a particularly type of nucleobase means that between 95-100% of the nucleobases present in the oligonucleobase are of that type.

“Separately synthesized” oligonucleobases are oligonucleobases synthesized independently of one another.

### SUMMARY OF THE INVENTION

It is an object of the invention to provide an oligonucleobase duplex mutational vector, called a “binary hybrid mutational vector” or “bHMV,” having a separate targeting strand and a mutator strand. The targeting strand contains a region of homology with a target gene sequence, and the mutator strand contains at least one base mismatch with respect to the targeting strand. The bHMVs are useful in altering specific eukaryotic or prokaryotic gene sequences. The separate targeting and mutator strands are also considered part of the invention.

It is an object of the invention to provide a method for producing the bHMVs and their component strands.

It is an object of the invention to provide methods of altering a specific eukaryotic and prokaryotic gene sequence using the bHMVs.

It is a further object of the invention to provide a method of selecting mutated cells from among a larger population of unmutated cells.

It is yet a further object of the invention to provide sets of bHMVs and/or the separate targeting or mutator strands for use in altering a specific gene sequence.

It is a still further object of the invention to provide kits containing the bHMVs and/or the separate targeting or mutator strands.

It is a still further object of the invention to provide bHMVs, modified bHMVs and bHMVs associated with a structure, all of which can be administered in vivo.

These and other objects of the invention will be apparent from the disclosure.

According to the present invention, a bHMV is provided comprising an oligonucleobase targeting strand and an oligonucleobase mutator strand, wherein said targeting strand comprises:

a) a 3′ end and a 5′ end; and

b) a targeting strand homologous region comprising a sequence of nucleobases homologous to a target gene sequence;

and wherein said mutator strand comprises:

c) a 3′ end and a 5′ end;

d) a complementary region comprising a sequence of nucleobases complementary to the targeting strand homologous region; and

e) a mismatch region located within the complementary region.

In preferred embodiments, the targeting strand comprises at least 70% ribo-type nucleobases and the mutator strand comprises at least 70% deoxyribo-type nucleobases. Most preferably, the ribo-type nucleobases are ribonucleotides, and the deoxyribo-type nucleobases are deoxynucleotides. In a particularly preferred embodiment, the bHMV comprises a targeting strand comprising at least 70% ribonucleotide bases and a mutator strand comprising at least 70% deoxyribonucleotide bases.

The present invention also provides separate targeting and mutator strands for use in constructing the bHMVs.

The invention is also directed to methods of producing the bHMVs and their component strands. There is thus provided a method of producing bHMVs comprising the steps of:

a) providing a separately synthesized targeting strand and mutator strand; and

b) mixing said targeting strand and said mutator strand such that the mutator strand complementary region containing the mismatch region forms a duplex with the targeting strand homologous region.

Upon hybridization, the 5′ end of the targeting strand is brought into juxtaposition with the 3′ end of the mutator strand, and the 5′ end of the mutator strand is brought into juxtaposition with the 3′ end of the targeting strand. The ends of the strands remain unligated, thus providing a duplex molecule with two nicks.

In another aspect, the invention provides oligonucleobase sets comprising targeting and mutator oligonucleobase strands according to the invention. The oligonucleobase sets comprise a common targeting strand and a plurality of mutator strands, wherein each mutator strand contains a unique mismatch region. In one embodiment, the common targeting strand is separately hybridized to each mutator strand to form an array of different bHMVs. The different bHMVs thus formed have identical target specificity, but will each introduce a different alteration into the target gene sequence. In another embodiment, the common targeting strand and each mutator strand are provided in separate containers, so that a selected mutator strand can be hybridized to the common targeting strand to form a bHMV.

In a further aspect, the invention provides kits for altering gene sequences in cells, comprising the sets of oligonucleobase strands described above. In one embodiment, the kit comprises:

1) a common targeting strand and a plurality of mutator strands as described above, wherein each mutator strand contains a unique mismatch region, and wherein the common targeting strand is hybridized separately to each mutator strand to form an array of different bHMVs; and

2) instructions and, optionally, reagents for introducing the bHMVs into cells.

In another embodiment, the kit comprises:

1) a common targeting strand and a plurality of mutator strands as described above, wherein each mutator strand comprises a unique mismatch region, and wherein the targeting strand and each mutator strand are provided in separate containers;

2) instructions and, optionally, reagents for hybridizing the mutator strand to the targeting strand to produce a bHMV; and

3) instructions and, optionally, reagents for introducing the bHMV into cells.

In another aspect, the invention provides a method of altering a target gene sequence, comprising the steps of:

a) providing a cell having a target gene sequence; and

b) introducing a bHMV as described above into said cell, so that the bHMV alters said target gene sequence.

In one aspect of the invention, the method is used to alter target sequences in eukaryotic cells. This method of altering specific eukaryotic gene sequences is particularly useful in the treatment of genetic diseases. In one embodiment, the bHMVs are used to repair mutations or introduce genetic alterations into any cell that can be removed from a subject's body, cultured and reimplanted into the subject. In particular, the bHMVs may be used to correct a deleterious mutation in cells from a human subject. The invention thus provides a method of treating a genetic disease, comprising:

a) providing a subject having at least one vector-repairable mutation associated with said disease;

b) placing a population of cells from said subject in culture, wherein said cells carry the mutation;

c) introducing a bHMV or mixture of bHMVs according to the invention designed to correct said vector-repairable mutation into said cells to form mutation-corrected cells; and

d) reimplanting said mutation-corrected cells into the subject.

In another aspect, a bHMV or mixture of bHMVs may be administered to a subject in vivo to effect an alteration in the genome of target cells. Any method for the in vivo adminstration of nucleic acids may be used to administer bHMVs. Although bHMvs may be administered in vivo without modification or without being associated with a structure, it is preferred that bHMVs are modified or associated with a structure so as to facilitate delivery of the bHMV to target cells. Preferred structures are liposomes, micelles and microcapsules. Thus, the present invention provides a method of altering a target sequence on a target cell in vivo, comprising the steps of:

a) providing a subject having a target cell with a target gene sequence; and

b) administering the binary hybrid mutational vector of claim 1 into said subject in vivo, so that said binary hybrid mutational vector alters the target gene sequence in the target cell,

wherein the bHMV is optionally modified or associated with a structure. bHMVs modified for in vivo adminstration, or bHMVs associated with a structure for in vivo administration, are also considered part of the present invention. The invention also provides pharmaceutical formulations of bHMVs for in vivo administration.

In another aspect, the bHMVs may also be used to mutagenize a population of cells to produce mutant cells having a selectable phenotype. Thus the invention provides a method of selecting mutated cells from among unmutated cells comprising the steps of:

a) providing a population of cells to be mutagenized;

b) transfecting said population of cells with at least one bHMV according to the invention to produce mutant cells having a selectable phenotype; and

c) isolating said mutants having the selectable phenotype with appropriate culture conditions.

In another aspect, the invention provides a method of constructing transgenic organisms, such as transgenic animals, particularly mammals, or plants. In one embodiment, transgenic animals are constructed by altering target gene sequences in embryonic stem (ES) cells, and obtaining chimeric animals by aggregation of the altered ES cells with normal blastocyst cells.

In another embodiment, a method of constructing a transgenic mammal is provided, comprising:

a) obtaining a mammalian gamete;

b) providing one or more bHMVs according to the invention for alteration of a target gene sequence in the gamete;

c) introducing said one or more bHMVs into the nucleus of the gamete to produce an altered gamete and fusing the altered gamete with another cell to form an altered zygote;

d) implanting the altered zygote into a female mammal capable of bearing the altered zygote to term; and

e) allowing the zygote to develop to term.

In another embodiment, a method of constructing a transgenic mammal is provided, comprising:

a) obtaining a mammalian zygote;

b) providing one or more bHMVs according to the invention for alteration of a target gene sequence in the zygote;

c) introducing said one or more bHMVs into the nucleus or pronucleus of the zygote to form an altered zygote;

d) implanting the altered zygote into a female capable of bearing the zygote to term; and

e) allowing the zygote to develop to term.

The invention also provides a method of constructing transgenic plants, comprising:

a) providing a plant cell or protoplast containing a target gene sequence;

b) introducing a bHMV according to the invention into said plant cell or protoplast, so that bHMV alters said target gene sequence to produce an altered plant cell or an altered protoplast; and

c) growing a mature plant from the altered plant cell or the altered protoplast.

### DETAILED DESCRIPTION OF THE INVENTION

For ease of illustration, the invention will at times be discussed in terms of ribonucleotide bases and deoxyribonucleotide bases. However, it is understood that the present invention is not limited to ribonucleotide and deoxyribonucleotide bases, and that ribo-type and deoxyribo-type nucleobases may also be employed.

The present invention provides a novel two-strand oligonucleobase duplex mutational vector, called a “binary hybrid mutational vector” or “bHMV,” which is useful in altering specific eukaryotic and prokaryotic gene sequences. The bHMV may be introduced into a cell by any method known to allow for the introduction of nucleic acids into cells. Without wishing to be bound by any theory, it is believed that the bHMV is engaged by the recombination/repair mechanisms of the target cell and directs the alteration of the target gene. The bHMVs exhibit a higher recombination rate than obtained with previous duplex mutational vectors. Without wishing to be bound by any theory, it is believed the bHMV is less conformationally constrained during recombination than previous duplex mutational vectors. The bHMVs may be used in any application requiring a specific alteration in the sequence of a target gene in a genome, particularly a eukaryotic genome. Preferred applications include, for example, the treatment of genetic diseases, construction of transgenic organisms and determination of the mechanisms or effects of targeted mutational changes in genes.

A bHMV comprises an oligonucleobase targeting strand and an oligonucleobase mutator strand. The nucleobases comprising a bHMV strand may comprise any nucleobase now known or to be developed that hybridizes to a complementary nucleobase.

Nucleobases comprise a base, which is a purine, pyrimidine, or a derivative or analog thereof. Nucleobases suitable for the present invention include, for example, nucleotides, nucleotoids, nucleosides, peptide nucleobases, the subunits of peptide nucleic acids and morpholine nucleobases. Nucleobases are either of the ribo- or deoxyribo-type. Ribo-type nucleobases contain pentosefliranosyl moieties wherein the 2′ carbon is substituted with a hydroxy, alkyl, or halogen. Deoxyribo-type nucleobases are nucleobases other than ribo-type nucleobases, and include nucleobases which do not contain a pentosefuranosyl moiety.

Nucleotoids are pentosefuranosyl-containing nucleobases having linkages that contain a phosphorous atom; e.g. phosphorothioates, phosphoroarnidates and methylphosphonates. Nucleotides are pentose-furanosyl-containing nucleobases that are linked by phosphodiester groups. The structure and synthesis of nucleotoids can be found in: phosphorothioates, Eckstein, F., Ann. Rev. Biochem., 1985, 54, 367; phosphoroamidates, Froehler, B. C., et al., Nucleic Acid Research, 1988, 16, 4831; methylphosphonates, Miller, P. S., et al., 1985, Biochimie, 1985, 67, 769, the entire disclosures of which are incorporated herein by reference. A method of producing oligonucleotoids having chiral-specific phosphorus-containing linkages is described in U.S. Pat. No. 5,212,295, the entire disclosure of which is incorporated herein by reference. Chirally-specific oligonucleotoids having the appropriately selected isomer hybridize to DNA with improved stability.

Nucleosides are pentosefuranosyl-containing nucleobases that are linked by non-phosphorus linkages. Nucleosides form duplexes with DNA that are at least as stable as DNA/DNA duplexes. The chemistries and methods for synthesizing oligonucleosides are described in the following: methylhydroxylamine linkages, Vasseur et al., J. Am. Chem. Soc. 1992, 114, 4006, U.S. Pat. Nos. 5,386,023 and 5,489,677; alkylenedioxy linkages, U.S. Pat. No. 5,223,618; and 3′-thioformacetal linkages, Jones et al., J. Org. Chem. 1993, 58, 2983. The aforementioned patents and publications are incorporated herein by reference in their entirety. Other nucleosides that can be used in synthesizing bHMV strands include, for example: carbamates, Stirchak et el., J. Org. Chem. 1987, 52, 4202; sulfonate and sulfonamide, Glemarec et al., Tetrahedron 1993, 49, 2287, Reynolds et al., J. Org. Chem. 1992, 57, 2983; sulfone, Huang, Z., J. Org. Chem. 1991, 56, 3869; sulfamate, Huie, E. M., et al., J. Org. Chem., 1992, 57, 4569; and diisopropylsilyl and silyl, Cormier and Ogilvie, Nucleic Acids Res. 1988, 16, 4583, Ogilvie & Cormier, Tetrahedron Lett. 1985, 26, 4159. The aforementioned publications are incorporated herein by reference in their entirety.

Nucleobases that lack a pentosefuranosyl moiety may be used as deoxyribo-type nucleobases in the practice of the invention. One type of non-pentosefuranosyl nucleobase may be prepared by replacement of the pentosefuranosyl phosphate moiety by a morpholino carbamate, Wang & Weller, Tetrahedron Lett., 1991, 32, 7385 the entire disclosure of which is incorporated herein by reference. Another type of non-pentosefuranosyl nucleobase are the subunits used to form peptide nucleic acids, in which the pentosefuiranosyl phosphate moiety is replaced by an aminoethylglycine moiety. Peptide nucleic acids (PNAs) are described in Egholm et al., J. Am. Chem. Soc., 1992, 114, 1895 and Huang, B. S. et al., J. Org. Chem., 1991, 56, 5006 and WO 92/20703 to Buchardt et al., and methods of making PNA/oligonucleotide chimeric polymers is described in WO 95/14706, the entire disclosures of which are incorporated herein by reference. Those skilled in the art understand that PNA can hybridize to DNA in either orientation, i.e., either end of a PNA can be the 3′ or 5′ end. Peffer, N. J., et al., 1993, Proc. Natl. Acad. Sci. 90:10648-52. When a peptide nucleobase is present in an oligonucleobase strand having pentosefuranosyl-containing nucleobases, the 3′ and 5′ ends of the strand are determined by the orientation of the pentosefuranosyl moieties or, if none are present in the chain having the peptide nucleobase, then the 3′ and 5′ ends of the strand are determined by the orientation of pentosefuranosyl nucleobases of the complementary strand.

The bHMV strands need not be of the same species of nucleobase; for example, an oligonucleoside targeting strand may be hybridized to an oligonucleotide mutator strand. Furthermore, an individual targeting or mutator strand may comprise different species of nucleobases, for example nucleosides and nucleotides.

The targeting strand contains a “targeting strand homologous region,” comprising a sequence of bases homologous to a target gene sequence. The “target gene sequence” is a specific sequence of a eukaryotic or prokaryotic gene to be altered. The mutator strand contains a “complementary region,” comprising a sequence of nucleobases complementary to the targeting strand homologous region. The mutator strand also contains a “mismatch region.” The mismatch region comprises a nucleobase sequence containing at least one base mismatch with respect to the nucleobase sequence of the targeting strand homologous region. The mismatch region is preferably centrally located within the mutator strand complementary region, and provides the alteration which is introduced into the target gene sequence. As used herein, “centrally located” means that the mismatch region is flanked by substantially equal portions of the mutator strand complementary region.

bHMVs containing a targeting strand homologous region comprising ribo-type nucleobases are particularly efficient for introducing a genetic alteration into the target gene sequence. In one embodiment, the targeting strand homologous region may comprise at least 70%, preferably at least 80%, and most preferably at least 90% ribo-type nucleobases. In a particularly preferred embodiment, the targeting strand homologous region consists essentially of ribo-type nucleobases.

In another embodiment, the entire targeting strand may comprise ribo-type nucleobases. For example, the targeting strand may comprise at least 70%, preferably at least 80%, and most preferably at least 90% ribo-type nucleobases. In a particularly preferred embodiment, the targeting strand consists essentially of ribo-type nucleobases.

Alteration of the target gene sequence is also particularly efficient when the mutator strand complementary region and mismatch regions comprise deoxyribo-type nucleobases. Thus, the mutator strand complementary and mismatch regions may comprise at least 70%, preferably at least 80%, and most preferably at least 90% deoxyribo-type nucleobases. In preferred embodiments, the mutator strand complementary and mismatch regions consist essentially of deoxyribo-type nucleobases.

In another embodiment, the entire mutator strand comprises deoxyribo-type nucleobases. For example, the mutator strand may comprise at least 70%, preferably at least 80%, and most preferably at least 90% deoxyribo-type nucleobases. In a particularly preferred embodiment, the mutator strand consists essentially of deoxyribo-type nucleobases.

The mismatch region may comprise bases different from those present in the target gene sequence (a substitution), bases in addition to those in the target gene sequence (an insertion), or the mismatch region may lack bases found in the target gene sequence (a deletion). The mismatch region may be as large as will support a stable bHMV duplex upon hybridization of the targeting and mutator strands, for example from 1 to about 5 bases in length. Particularly preferred are mismatch regions of 1, 2 or 3 bases. Two or more mismatch regions may be contained in the mutator strand complementary region, as long as the mismatch regions are separated from one another by sufficient number of complementary bases to a form a stable duplex. For example, two one-base mismatch regions separated by 1-30 bases may be contained in a targeting strand homologous region of 40 bases in length.

The targeting strand homologous region may be at least 10 bases in length, and may be as large as about 150 bases. Preferably, the targeting strand homologous region is between 25-60 bases, and most preferably about 40 bases. The length of the mutator strand complementary region, which includes the mismatch region, is comparable to the length of the targeting strand homologous region. It is understood that the total length of mutator strand complementary and mismatch regions may differ from the length of the targeting strand homologous region to the extent the mismatch region differs from the target gene sequence. For example, if the mismatch region comprises an insertion or deletion of a certain number of bases, then the overall length of the mutator strand complementary region (containing the mismatch region) will differ from the length of the targeting strand homologous region by the same number of bases.

At least one of the ribo-type nucleobases found in either the targeting or mutator strands may be nuclease-resistant. Suitable nuclease resistant ribo-type nucleobases may be 2′AX-nucleosides, 2′AX-nucleotoids and 2′AR-nucleotides, wherein:

A is oxygen or a halogen (preferably fluorine, chlorine or bromine);

X is hydrogen or C₁₋₆ alkyl; and

R is C₁₋₆ alkyl;

provided that when A is a halogen, then X and R are omitted.

The carbon chain of the C₁₋₆ alkyl moiety may be straight-chain or branched. Straight-chain alkyl moieties are preferred. Preferred nuclease resistant ribo-type nucleobases are 2′-O methyl ribo-type nucleobases, and particularly preferred are 2′-O methyl ribonucleotides.

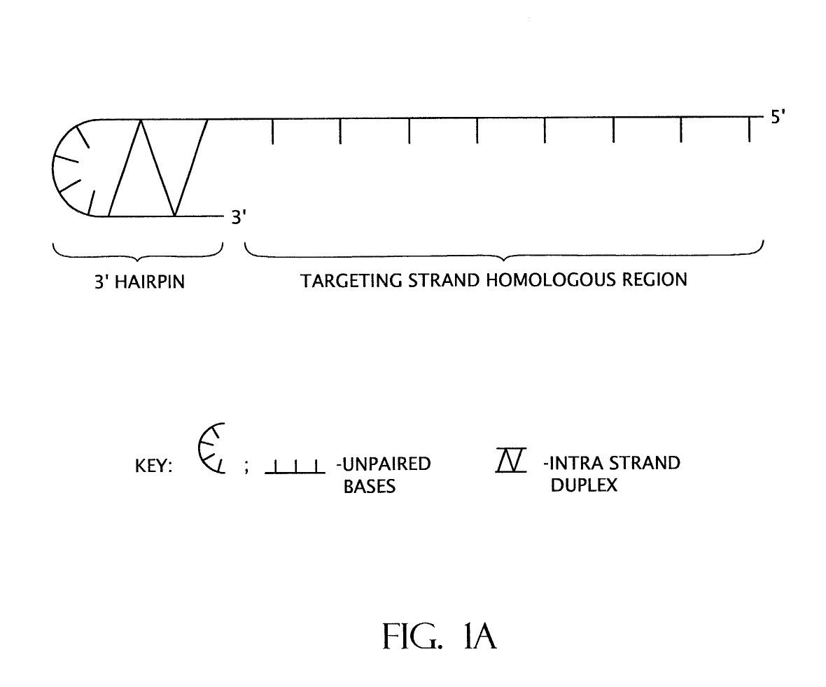

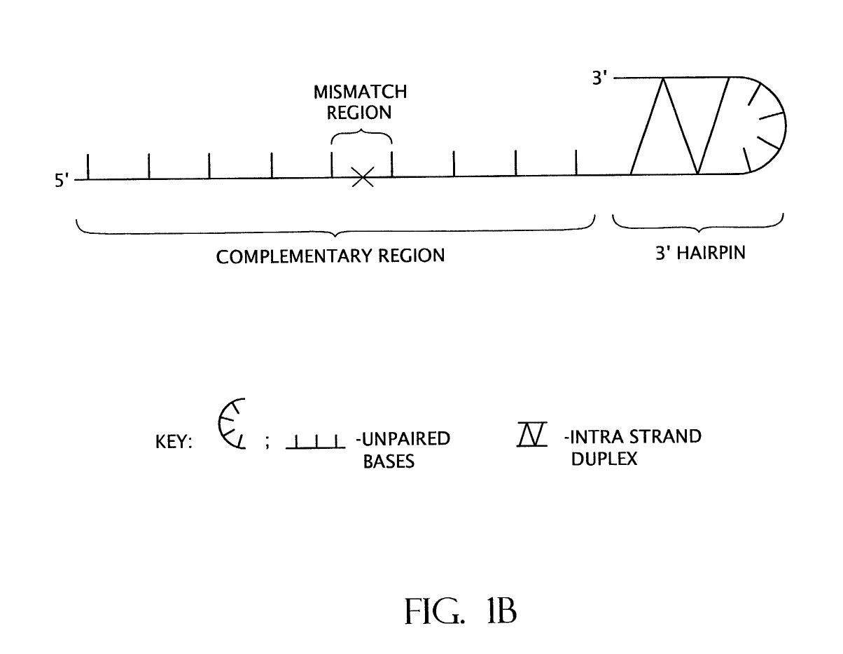

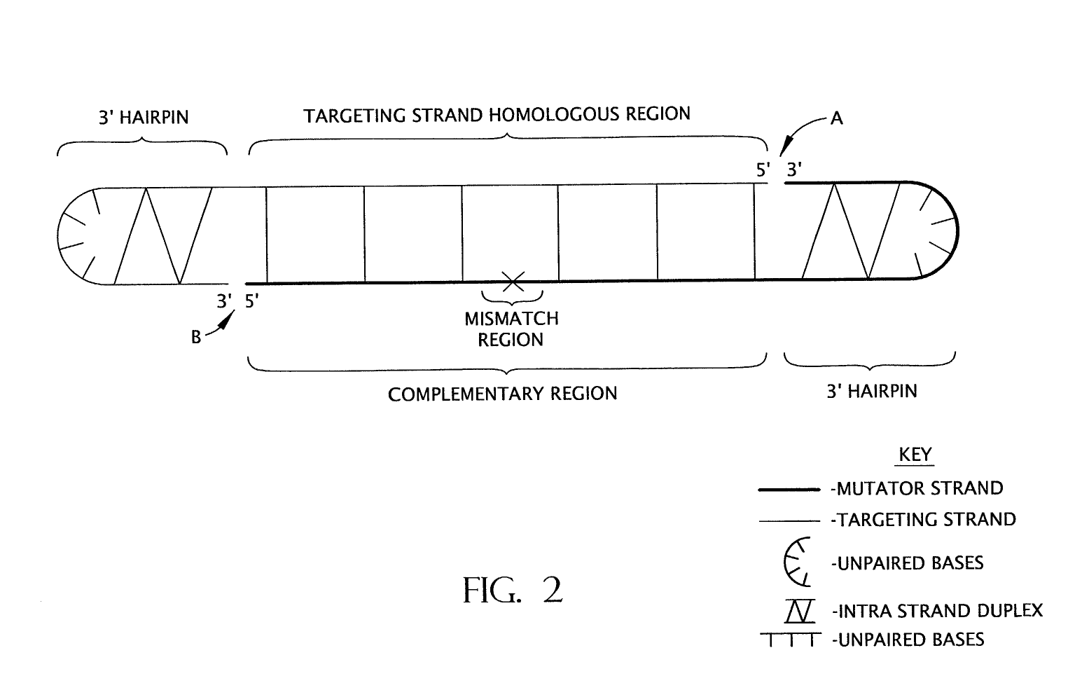

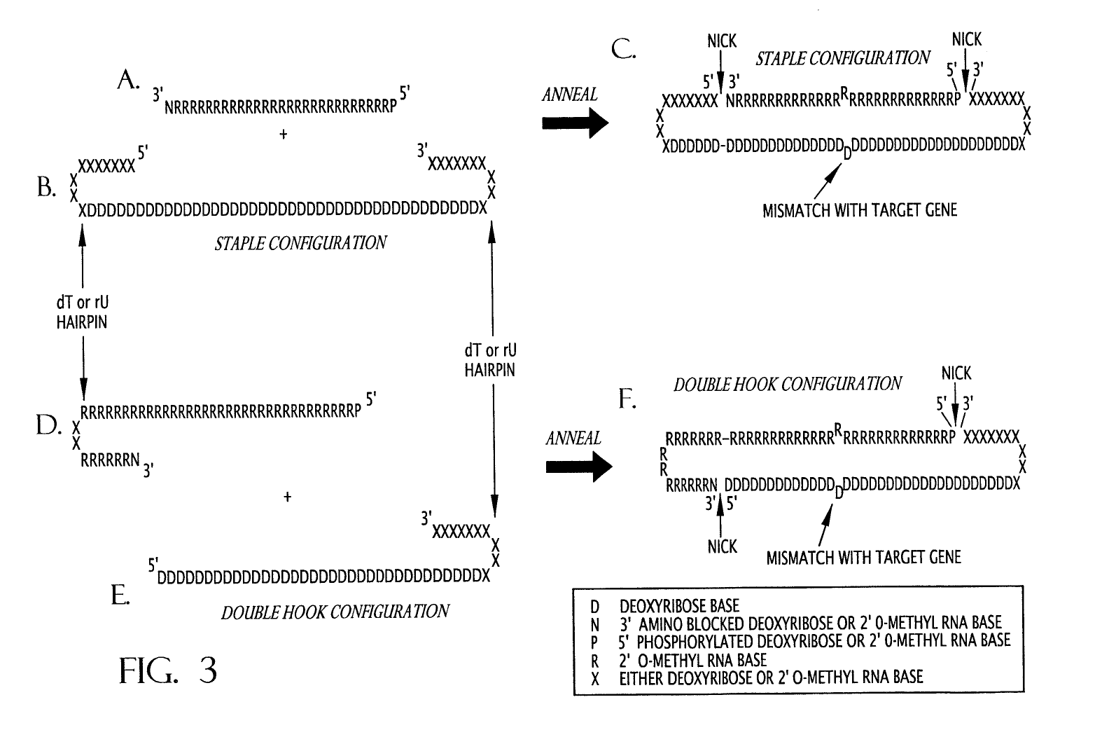

In one embodiment, the targeting and mutator strands are similar in secondary structure. For example, both targeting and mutator strands may contain hairpins at their 3′ ends and single-stranded regions adjacent to the hairpins extending in the 5′ direction along the respective strands. On the targeting strand, the single-stranded region is the targeting strand homologous region. On the mutator strand, the single-stranded region contains the mutator strand complementary region and the mismatch region. In this embodiment, the hairpins on both the targeting and mutator strands consist of an intra-strand duplex region of complementary nucleobases linked by a region of contiguous unpaired bases; see FIGS. 1A and IB. Preferably, the intra-strand duplex region consists of 4-8 nucleobase pairs and the region of contiguous unpaired nucleobases consists of 3-10 nucleobases. The complementary nucleobases in the intra-strand duplex region may comprise a series of alternating guanine and cytosine bases, commonly called a “GC clamp” because of the higher stability conferred upon the duplex region by the G:C base pairs. When the targeting and mutator strands are mixed together, the regions of designed inter-strand complementarity (i.e. the targeting strand homologous region and the mutator strand complementary region) will hybridize to form an inter-strand duplex; see FIG. 2 and FIG. 3B. The inter-strand duplex begins at the nucleobases in the targeting strand homologous region which are immediately adjacent to the 3′ hairpin and extend up through the 5′-most nucleobase on the targeting strand. With reference to the mutator strand, the inter-strand duplex begins at the nucleobases in the complementary region which are immediately adjacent to the 3′ hairpin and extend up through the 5′-most nucleobase on the mutator strand. Through hybridization, the 5′ end of the targeting strand is brought into juxtaposition with the 3′ end of the mutator strand, and the 5′ end of the mutator strand is brought into juxtaposition with the 3′ end of the targeting strand. These ends are not ligated together, and remain as “nicks” in the duplex vector (see positions A and B of FIG. 2). The presence of the mismatch region within the mutator strand complementary region will not prevent hybridization of the two strands. Preferably, the 3′ end of each strand is blocked from ligation with the 5′-end of the other strand. Blocking ligation of 3′ end of the targeting strand can be accomplished, for example, by the substitution of blocking groups for the 3′-OH of the 3′ terminal base. Suitable blocking groups include, for example, straight-chain alkyl moieties with a terminal amino, hydroxyl or halo functionality. A preferred blocking group is propyl amine. Blocking of ligation at the 3′ ends may also be accomplished by placing a moiety incapable of ligation at a 3′ terminus, for example a subunit of a peptide nucleic acid or a dideoxynucleotide.

In another embodiment, one strand is longer than the other and contains a hairpin at each end of the molecule with a stretch of single-stranded nucleobases in between the hairpins. The other, shorter strand has no secondary structure (i.e., is completely single-stranded), and is complementary to the single-stranded sequence of the longer strand located between the two hairpins. Thus, hybridization of the two strands brings the 3′ and 5′ ends of the shorter strand into juxtaposition with the 5′ and 3′ ends of the longer strand, respectively. The 5′ and 3′ ends of the longer strand delineate the ends of the hairpin regions (see FIG. 3A). It is preferred that the longer strand comprise the mutator strand and the shorter strand comprise the targeting strand. In embodiments where the mutator strand is the longer strand, the mutator strand complementary region is located in the single-stranded region between the hairpins. It is also preferred that the targeting strand homologous region on the shorter strand comprises substantially all of the single-stranded region (i.e., the targeting strand homologous region is coextensive with the targeting strand). The hairpin conformation and composition on the mutator strand is as described above.

A preferred targeting strand is characterized by:

a) a 3′-terminal ribonucleotide which is optionally blocked from ligation;

b) a 3′ hairpin formed by an intra-strand duplex region of 4-8 ribonucleotide base pairs linked by a region of four contiguous unpaired uracil ribonucleotides; and

c) a targeting strand homologous region located adjacent to the 3′ hairpin and extending in the 5′ direction along said strand, comprising a sequence of ribonucleotide bases homologous to a

target gene sequence.

A preferred mutator strand is characterized by:

a) a 3′ hairpin formed by an intra-strand duplex region of 4-8 deoxyribonucleotide base pairs linked by a region of four contiguous unpaired thymine deoxyribonucleotides;

b) a complementary region located adjacent to the 3′ hairpin and extending in the 5′ direction along said strand, comprising a sequence of deoxyribonucleotide bases complementary to the targeting strand homologous region; and

c) a mismatch region located within the complementary region.

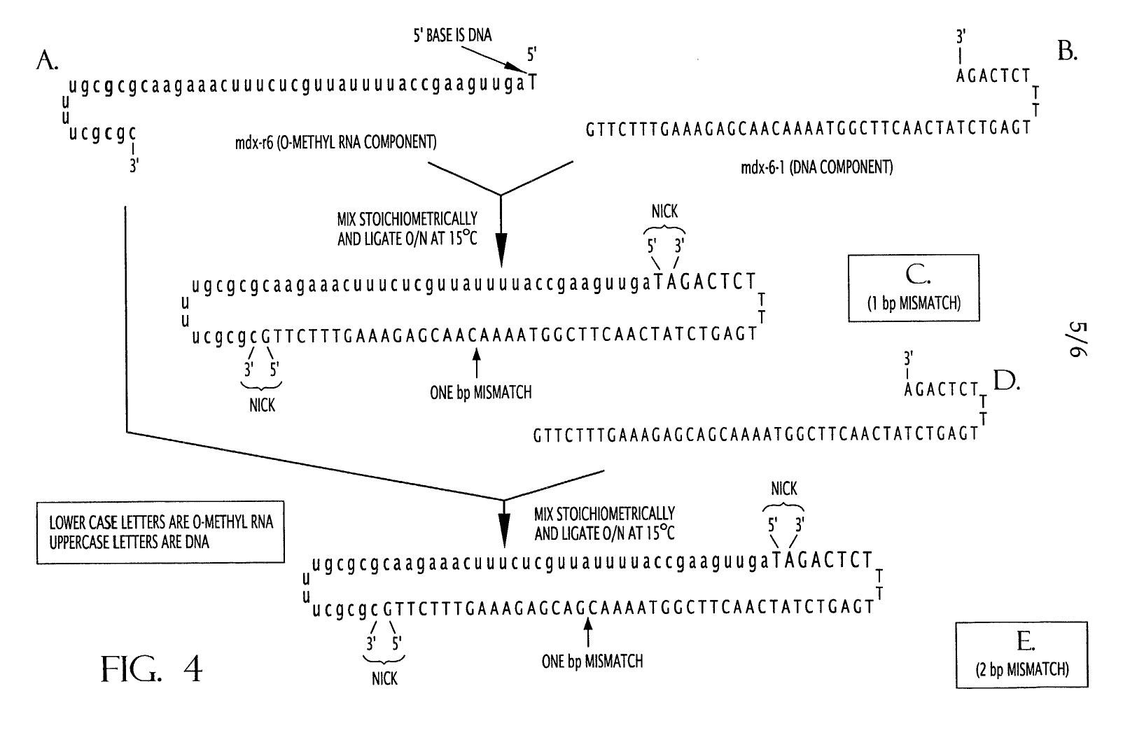

Examples of targeting and mutator strands, and the bHMVs produced by the hybridization of these strands, are shown in FIG. 4. Molecule A in FIG. 4 is targeting strand mdx-r6 (SEQ ID NO:1). Molecules B and D in FIG. 4 are mutator strands mdx-6-1 (SEQ ID NO:2) and mdx-6-2 (SEQ ID NO:3), respectively. Molecules C and E in FIG. 4 are, respectively, the bHMVs produced by hybridization of mutator strands mdx-6-1 and mdx-6-2 to targeting strand mdx-r6. Mutator strand mdx-6-1 contains a one-base mismatch with respect to targeting strand mdx-r6, and mutator strand mdx-6-2 has a two-base mismatch with respect to targeting strand mdx-r6.

It is a further object of this invention to provide a method of producing bHMVs. The targeting and mutator strands are synthesized separately and assembled after separate synthesis by mixing the strands and allowing them to hybridize.

The bHMV strands may be synthesized by any technique used to synthesize oligonucleobases. Solid-phase synthesis is the preferred technique for synthesizing strands up to about 100 bases in length. Alternatively, strand subsegrnents greater than about 50 bases in length may be obtained by solid phase synthesis and ligated by liquid phase techniques well known to those skilled in the art. Wosnick, M. A., 1987, Gene 60:115-117. Separate synthesis of the targeting and mutator strands allows greater vector purity since the two strands will be about half as long as a contiguously synthesized vector, and can therefore be synthesized with simpler chemistry. In embodiments where the targeting strand is composed of ribo-type nucleobases and the mutator strand is composed of deoxyribo-type nucleobases, the synthesis of each strand can proceed with relative ease and at a reduced cost because there is no need to mix synthetic chemistries. The two separately synthesized strands may be further purified prior to assembly, contributing to the purity of the final product.

The bHMV is assembled by mixing the separately synthesized targeting and mutator strands under conditions which permit hybridization of the targeting strand homologous region to the mutator strand complementary and mismatch regions. The presence of the mismatch region will not prevent the formation of an inter-strand duplex between the targeting and mutator strands. Preferably, the two strands are mixed stochiometrically, but may be mixed in any ratio. The process of oligonucleobase hybridization is well understood by those skilled in the art, and the conditions under which any set of targeting and mutator strands will hybridize can be readily ascertained. For a general discussion of oligonucleobase hybridization techniques, see Sambrook J, et al. (1989) Molecular Cloning: A Laboratory Manual, 2nd ed., Cold Spring Harbor Laboratory Press, which is incorporated herein by reference.

A further advantage of the binary (i.e., separate strand) synthesis method is the capacity to easily synthesize different mutator strands and hybridize them to a targeting strand. A targeting strand containing, for example, containing 2′-O-methyl ribo-type oligonucleobases is more expensive to synthesize than a mutator strand composed of deoxyribo-type nucleobases. One can synthesize a single targeting strand containing 2′-O-methyl ribo-type oligonucleobases and a plurality of less expensive mutator strands, wherein each mutator strand has a unique mismatch region. The mutator strands are then hybridized with aliquots of the targeting strand to form an array of separate bHMVs.

It is also an object of this invention to provide oligonucleobase sets comprising targeting and mutator strands. The oligonucleobase sets comprise a common targeting strand and a plurality of mutator strands, wherein each mutator strand contains a unique mismatch region. In one embodiment, a common targeting strand is hybridized separately to each mutator strand to form an array of different bHMVs. The different bHMVs thus formed have identical target specificity, but will introduce different changes into the target gene sequence. In another embodiment, a common targeting strand and a plurality of mutator strands are each provided in separate containers. Each mutator strand contains a unique mismatch region. A selected mutator strand may be hybridized to the common targeting strand to form a bHMV according to the invention. Again, each bHMV formed has identical target specificity, but will introduce a different alteration into the target gene sequence. The advantage to providing the targeting and mutator strands in different containers is that an investigator may choose which mutator strand to employ in forming a bHMV.

Kits are also provided containing the oligonucleobase sets described above. In all kit embodiments, the hybridized or individual strands are provided in a form suitable for shipping and storage, for example as a lyophilized powder or ethanol precipitate. Reagents and instructions for hybridizing targeting and mutator strands to form bHMVs, or for using the bHMVs to alter a target gene sequence, are optionally included in the kits. As used herein, “reagents” means the chemicals, buffers, solutions, and the like needed to effect a particular technique.

The present invention also encompasses methods of using bHMVs to alter a target gene sequence.

In one embodiment, the bHMVs may be used to introduce an alteration into the genome of an embryonic stem (ES) cell. ES cells are pluripotent cells that can be directed to differentiate into any cell type. Under certain conditions, for example as described in Thomson et al. (1998) Science 282:1145-1147 and Reubinoff et al. (2000) Nat. Biotechnol. 18:399-404 (both incorporated herein by reference), pluripotential ES cells can be propagated indefinitely in vitro and still maintain the capacity for differentiation into a wide variety of somatic and extraembryonic tissues. The present invention thus provides methods of transfecting ES cells with one or more bHMVs to alter a target gene sequence in the ES cell genome. Altered ES cells may be used for a variety of purposes, including the in vitro screening of chemical compounds for toxicological effects and pharmacological activity, providing donor tissue in transplantation procedures, and the creation of somatic cell lineages. ES cells useful in the present invention may be derived from any animal, preferably mammals; for example mice, human and non-human primates.

In another embodiment, the bHMVs may be used to repair mutations or introduce genetic alterations into any cell that can be removed from a subject's body, cultured and reimplanted into the subject. Suitable subjects include any animal, for example a mammal, or a plant. Preferably, the subject is a human.

In particular, the bHMVs may be used to correct a deleterious mutation in cells from a subject. The deleterious mutation can, for example, cause the production of an abnormal gene product or the altered expression of a normal gene product in the cells. Suitable cells are those which may be transiently placed in a cell culture with or without growth, and subsequently reimplanted into the subject. In one embodiment, the target cells removed from the subject comprise hematopoietic cells, particularly hematopoietic stem cells. As used herein, “hematopoietic cells” includes both precursors and mature cells of the erythroid, lymphoid monocytoid (macrophage) and granulocytic lineages. As used herein “hematopoietic stem cells” (“HSC”) includes all cells found in either the bone marrow or the peripheral blood that can repopulate the bone marrow space and produce progeny of the hematopoietic lineage. In another embodiment, the target cells removed from the subject comprise hepatocytes, particularly hepatic reserve (stem) cells. Techniques for the removal, culture and reimplantation of hepatocytes, in particular of hepatic reserve (stem) cells, are described in patent publication WO94/08598 to Naughton GB and Sibanda B, the entire disclosure of which is incorporated herein by reference. In a further embodiment, the target cells removed from the subject comprise embryonic stem (ES) cells. ES cells transfected with a bHMV may be directed to differentiate into the desired somatic or extraembryonic cell type before implantation, or implanted in an undifferentiated state and allowed to differentiate in situ.

The present invention therefore provides a method of treating genetic diseases in which a population of cells from subject are grown in culture and exposed to a bHMV or mixture of bHMVs designed to correct a mutation associated with the disease. The transfected cells are then reimplanted into the subject, where they express an altered phenotype which alleviates the effects of the disease.

A bHMV or mixture of bHMVs may be administered to a subject in vivo to effect an alteration in the genome of target cells. Any method for the in vivo adminstration of nucleic acids may be used to administer bHMVs. Although bHMvs may be administered in vivo without modification or without being contained within a structure, it is preferred that bhMVs be modified or associated with a structure so as to facilitate delivery of the bHMV to target cells. Target cells include hematopoietic cells, hematopoietic stem cells, hepatocytes, hepatic reserve cells, tumor or neoplastic cells, cells of the lung, or any other cell which carries a target gene sequence to be altered. Any route of administration may be used, for example including oral, enteral or parenteral. Oral administration includes, but is not limited to, inhalation through the mouth or nose. Parenteral administration includes, but is not limited to, intravascular administration (e.g., intravenous bolus injection, intravenous infusion, intra-arterial bolus injection, intra-arterial infusion and catheter instillation into the vasculature), peri- and intra-target tissue injection (e.g., peri-tumoral and intra-tumoral injection), direct application to the target tissue (e.g., by a catheter or other placement device), subcutaneous injection or deposition including subcutaneous infusion (e.g., by osmotic pumps) and intrathecal administration.

Suitable modifications of bHMVs for in vivo administration include, but are not limited to, the following. Such modified bHMVs are considered part of the present invention.

For example, the bHMV may be conjugated to poly(L-lysine) to increase cell penetration upon in vivo administration. Poly(L-lysine) nucleic acid conjugates are described by Lemaitre et al., Proc. Natl. Acad. Sci. USA, 84, 648-652 (1987), and the techniques in this article can be readily adapted for use with bHMVs by one of ordinary skill in the art. The procedure requires a 3′-terminal ribonucleotide. The resulting aldehyde groups are then randomly coupled to the epsilon-amino groups of lysine residues of poly(L-lysine) by Schiff base formation, and then reduced with sodium cyanoborohydride. This procedure converts the 3′-terminal ribose ring into a morpholine structure. bHMVs may also be conjugated with appropriate ligand-binding molecules to facilitate delivery to target cells upon in vivo adminstration. For example, bHMVs may be conjugated for therapeutic administration to targeting group which recognizes cell-surface molecules, such as according to International Patent Application WO 91/04753. The targeting group may comprise, for example, an antibody against a cell surface antigen, an antibody against a cell surface receptor, a growth factor having a corresponding cell surface receptor, an antibody to such a growth factor, or an antibody which recognizes a complex of a growth factor and its receptor. Methods for conjugating targeting groups to oligonucleotides are detailed in WO 91/04753. In particular, the growth factor to which the bHMV may be conjugated may comprise transferrin or folate. Transferrin-polylysine-bHMV complexes or folate-polylysine-bHMV complexes may be prepared for uptake by cells expressing high levels of transferrin or folate receptor. The preparation of transferrin complexes as carriers of oligonucleotide uptake into cells is described by Wagner et al., Proc. Natl. Acad. Sci. USA 87, 3410-3414 (1990). Cellular delivery of folate-macromolecule conjugates via folate receptor endocytosis, including delivery of an oligonucleotide, is described by Low et al., U.S. Pat. No. 5,108,921. Also see, Leamon et al., Proc. Natl. Acad. Sci. 88, 5572 (1991).

The present invention also provides pharmaceutical formulations comprising the bHMVs to be administered in vivo. Pharmaceutical formulations of the present invention are characterized as being at least sterile and pyrogen-free. As used herein, “pharmaceutical formulations” include formulations for human and veterinary use.

Examples of pharmaceutical formulations include bHMVs mixed with a physiologically acceptable carrier medium to form solutions, suspensions or dispersions. Preferred physiologically acceptable carrier media are water or normal saline. Pharmaceutical formulations can also include conventional pharmaceutical excipients and/or additives. Suitable pharmaceutical excipients include, for example, stabilizers, antioxidants, osmolality adjusting agents, buffers, and pH adjusting agents. Suitable additives include, for example, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA, CaNaDTPA-bisamide), or, optionally, additions (e.g. 1 to 50 mole percent) of calcium or sodium salts (for example, calcium chloride, calcium ascorbate, calcium gluconate or calcium lactate). Pharmaceutical formulations according to the present invention may be prepared in a manner fully within the skill of the art.

bHMVs may also be delivered in vivo to target cells by a structure. Examples of structures useful for carrying bHMV to target cells within a subject include liposomes, micelles, and microcapsules. The structure serves to more effectively direct the bHMV to target cells. The structure may also protect the bHMV from degradation or clearance by the body. Structures useful in the present invention may be modified to affect their biodistribution, for example by having opsonization inhibition moieties or targeting groups bound to the surface of the structure. In a preferred embodiment, a structure has both opsonization inhibition moieties and targeting groups bound to its surface.

In one embodiment, the structure is a liposome. As used herein, “liposome” refers to a generally spherical entity formulated from amphiphilic compounds which is characterized by the presence of at least one internal void. Preferred amphiphilic compounds are lipids. In any given liposome, the amphiphilic compounds may be in the form of a one or more monolayers or bilayers. Where a liposome comprises more than one mono- or bilayer, the mono- or bilayers are generally concentric. The liposomes described herein include such entities commonly referred to as liposomes, bubbles, microbubbles, microspheres, vesicles and the like. Thus, the amphiphilic compounds may be used to form a unilamellar liposome (comprised of one monolayer or bilayer), an oligolamellar liposome (comprised of about two or about three monolayers or bilayers) or a multilamellar liposome (comprised of more than about three monolayers or bilayers). As used herein, the term “liposome” also refers to multivesicular liposomes, which are liposomes comprising multiple non-concentric voids. For examples of multivesicular liposomes and methods of their preparation, see U.S. Pat. No. 5,993,850 of Sankaram et al. and U.S. Pat. No. 5,997,899 of Ye et al., the disclosures of which are herein incorporated by reference in their entirety. Multivesicular liposomes are especially useful for sustained or timed release of the bHMVs.

The internal voids of the liposomes may be filled with a liquid, solid or gas, or any combination thereof. For example, liposome voids may be filled with one or more of an aqueous liquid, a gas, a gaseous precursor, and/or a solid or solute material.

Thus in one aspect of the present invention, bHMVs are carried by liposomes. As used herein, “carried by liposomes” means the bHMV is embedded within the wall of the liposome, encapsulated in the liposome or attached to the liposome. As used herein, “attached to” means that the bHMV is associated in some manner to the inside and/or the outside wall of the liposome, such as through a covalent or ionic bond, or other means of chemical or electrochemical linkage or interaction. As used herein, “encapsulated in” means that the bHMV is located in the internal liposome void. As used herein, “embedded within” means the bHMV is within the liposome wall. Thus, the bHMV may be positioned variably, such as, for example, entrapped within the internal void of the liposome, incorporated onto the internal/external surfaces of the liposome and/or enmeshed within the liposome structure itself.

Preparation of liposomes carrying bHMVs is within the skill of those in the art. For example, a bHMV may be made lipophilic or amphiphilic; e.g., by conjugating the bHMV with one or more lipophilic or amphiphilic groups. Efficient embedding of the lipophilic or amphiphilic bHMV within the liposome wall may be achieved by preparing a mixture of liposome-forming material and the lipophilic or amphiphilic bHMV, e.g., in a dried film, and hydrating the mixture. This procedure will form liposomes with lipophilic or amphiphilic bHMV embedded predominantly in the wall of the liposomes. Useful techniques for incorporating amphiphilic compounds into liposome membranes are disclosed, e.g., in Grant et al., Magn. Res. Med., 11:236-243 (1989); Kabalka et al., Magn. Res. Med., 8:89-95 (1988); and Hnatowich et al., J. Nucl. Med., 22:810-816 (1981), the disclosures of which are herein incorporated by reference in their entirety.

Alternatively, the amphiphilic material used in forming the liposomes may be conjugated with the bHMV prior to liposome formation. Liposomes formed with amphiphilic material conjugated with a bHMV will carry the bHMV attached to both the inner and outer liposome surfaces. The bHMV may also be conjugated to the liposome after it has been formed, which will result in the bHMV being carried only on the outside surface of the liposome.

bHMVs which are not modified by conjugation to amphipilic or lipophilic groups are typically hydrophilic. Passive loading may be employed for preparing liposomes with encapsulated hydrophilic bHMVs. In this case, the bHMV is usually dissolved in the aqueous medium used to hydrate a film of liposome-forming material. Typically, the aqueous medium containing the film is sonicated to form liposomes encapsulating the bHMV dissolved in the aqueous solution. Encapsulation efficiencies of between about 5-20% are typically obtained, with the remainder of the bHMV being in the bulk aqueous phase. An additional processing step for removing non-encapsulated bHMV is therefore usually required. For other techniques by which hydrophilic materials are encapsulated in liposomes; see e.g. Bangham et al. J. Mol. Biol., 13:238-252 (1965); D. Papahadjopoulos and N. Miller. Biochim. Biophys. Acta, 135:624-638 (1967); Batzri and Kom. Biochim. Biophys. Acta, 2981015 (1973); Deamer and Bangham. Biochim. Biophys. Acta, 443:629-634 (1976); Papahadjopoulos et al. Biochim. Biophys. Acta, 394:483491 (1975); German Pat. No. 2,532,317; and U.S. Pat. Nos. 3,804,776; 4,016,100 and 4,235,871, the disclosures of which are herein incorporated by reference in their entirety.

A more efficient method for encapsulating hydrophilic compounds, involving reverse evaporation from an organic solvent, has been reported; see Szoka, F., Jr., et al., (1980) Ann. Rev. Biophys. Bioeng. 9:467 the disclosure of which is herein incorporated by reference in its entirety. In this approach, a mixture of hydrophilic bHMV and liposome-forming lipids are emulsified in a water-in-oil emulsion, followed by solvent removal to form an unstable lipid-monolayer gel. When the gel is agitated, typically in the presence of added aqueous phase, the gel collapses to form oligolamellar liposomes with high (up to 50%) encapsulation of the bHMV.

The liposomes of the present invention deliver the carried bHMV at or near target cells. Liposomes carrying bHMVs may fuse with a target cell, be taken up by a target cell, or release their contents outside a target cell. Liposomes are particularly effective at delivering bHMVs to tumor cells.

The liposomes useful in the present invention may be any size. Particularly preferred liposomes are those which are small enough to pass through the pulmonary capillary bed; i.e. those with a diameter of approximately 8 microns. However, the liposomes may have a diameter of about 0.1 to about 2,000 microns. Preferred liposomes are those having a diameter of about 100 to 1,000 microns, others having a diameter of about 10 to 100 microns, and still others having a diameter of about 1 to 100 microns. A preparation of liposomes typically has a distribution of sizes. A liposome preparation in a range of about 0.1 to 10 microns with a size distribution within less than about a 20% standard deviation of the average diameter is preferred. A liposome preparation in a range of about 0.1 to 10 microns with a size distribution within about 10% of the average diameter is still more preferred.

Delivery of a bHMV may be enhanced by the external application of energy to the delivery site, for example by the application of ultrasound to the tumor site. The ultrasound energy will either enhance the fusion of the liposomes with the target cell, or cause the liposomes to break, thus exposing target cells to the bHMV. Examples of ultrasound-mediated delivery of nucleic acids to target cells via liposomes are given in U.S. Pat. No. 6,056,938 of Unger et al., the entire disclosure of which is herein incorporated by reference.

Preferred liposomes are “modified liposomes.” Modified liposomes carry components on their outer surface that affect biodistribution, for example opsonization inhibiting moieties or targeting groups with a specific affinity for a target cell. A modified liposome can comprise opsonization inhibiting moieties and targeting groups.

Opsonization-inhibiting moieties are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization inhibiting hydrophilic polymers form a protective surface layer which significantly decreases the uptake of the liposomes by the macrophage-monocyte system (MMS) and reticuloendothelial system (RES), e.g., as described in U.S. Pat. No. 4,920,016, which is herein incorporated by reference in its entirety. Liposomes modified with opsonization inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example solid tumors, will efficiently accumulate these liposomes; see Gabizon, et al., P.N.A.S., USA, 18:6949-53 (1988). In addition, the reduced uptake by the RES lowers the toxicity of stealth liposomes carrying bHMVs by preventing significant accumulation in the liver and spleen. Thus, liposomes that are modified with opsonization inhibition moieties deliver the bHMV to tumor cells, whereupon the bHMV may effect the desired alteration of the target gene sequence.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives, e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM,. Copolymers of PEG, methoxy PEG, or methoxy PPG, or derivatives thereof, are also suitable. In addition, the opsonization inhibiting polymer may be a block copolymer of PEG and either a polyamino acid, polysaccharide, polyamidoamine, polyethyleneamine, or polynucleotide. The opsonization inhibiting polymers may also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

The opsonization inhibiting polymer may be bound to the liposome membrane by any one of numerous well-known techniques. For example, an N-hydroxysuccinimide ester of PEG may be bound to a phosphatidyl-ethanolamine lipid-soluble anchor, and then bound to a membrane. Similarly, a dextran polymer may be derivatized with a stearylamine lipid-soluble anchor via reductive amination using Na(CN)BH₃ and a solvent mixture such as tetrahydrofuran and water in a 30:12 ratio at 60° C.

To obtain a liposome that is targeted for a specific target cell, a targeting group is bound to the outer surface of the liposome. As used herein, a targeting group is “bound” to a liposome membrane when it is chemically or physically attached to the membrane, e.g., by the intercalation of a lipid-soluble anchor into the membrane itself, or by binding directly to active groups of membrane lipids.

For example, the carbohydrate portion of the liposome membrane is oxidized, e.g., by exposure to sodium metaperiodate to yield aldehyde groups, which are highly reactive and will bind the target group to the membrane. In addition, the target group may be linked to a lipid-soluble anchor, and the anchor is then intercalated into the liposome membrane. These and other methods of binding targeting groups to liposome membranes are described in U.S. Pat. No. 4,483,929, the disclosure of which is herein incorporated by reference in its entirety.

Suitable targeting groups include compounds selected or designed to target a target cell; for example polyclonal or monoclonal antibodies, fragments of antibodies, chimeric antibodies, an enzyme or enzyme substrate, a lectin, a saccharide ligand of a lectin, and small molecule ligands. It is understood that these targeting groups, and the specific examples of such targeting groups listed below, may also be conjugated directly to the bHMVs.

A preferred small molecule ligand is the E. coli heat stable enterotoxin ST, which specifically targets cells of colorectal origin, such as metastasized colorectal cancer cells. The E. coli heat stable enterotoxin ST is described in Waldman, U.S. Pat. No. 5,518,888, the disclosure of which is herein incorporated by reference in its entirety.

Preferred antibodies include antibodies to tumor-associated antigens. Specific examples include, for example, B72.3 antibodies (described in U.S. Pat. Nos. 4,522,918 and 4,612,282) which recognize colorectal tumors, 9.2.27 anti-melanoma antibodies, D612 antibodies which recognize colorectal tumors, UJ13A antibodies which recognize small cell lung carcinomas, NRLU-10 (Tfs-2) antibodies which recognize small cell lung carcinomas and colorectal tumors, 7E11C5 antibodies which recognize prostate tumors, CC49 antibodies which recognize colorectal tumors, TNT antibodies which recognize necrotic tissue, PRIA3 antibodies, which recognize colon carcinoma (see Richman, P. I. and Bodmer, W. F. (1987) Int. J. Cancer Vol. 39, pp. 317-328), ING-1 and other genetically engineered antibodies, which are described in WO-A-90/02569, B174 antibodies (developed at Biomira, Inc. of Edmonton, Canada), which recognize squamous cell carcinomas, B43 antibodies which are reactive with certain lymphomas and leukemias, and other antibodies which may be of particular interest. All references cited in this paragraph are herein incorporated by reference in their entirety.

As many solid tumors are surrounded by an area of neovascularization, other suitable targeting groups include antibodies directed to cell surface antigens of neovascular endothelium (e.g., anti-CD105 antibodies) or ligands with affinity for cell surface receptors of neovascular endothelium.

Also preferred are antibodies directed to markers of vascular restenosis, for example cell surface antigens of vascular smooth muscle cells (VSMCs). VSMCs are a major component of restenotic lesions seen in patients undergoing treatment for coronary artery disease. Other useful antibodies include those directed to the product of the IRT-1 gene, as described in WO 99/34814, the disclosure of which is herein incorporated by reference in its entirety.

Pharmaceutical formulations of liposomes carrying bHMVs may be formulated as described above, but may contain additional emulsifiers and/or viscosity modifiers designed to keep the liposomes in suspension. Suitable viscosity modifiers include, for example, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, propylene glycol alginate, silicon dioxide, sodium alginate, tragacanth, and xanthan gum. Suitable emulsifiers include, for example, poloxamers and their derivatives, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, and emulsifying wax. Pharmaceutical formulations according to the present invention comprising liposomes may be prepared in a manner fully within the skill of the art.

In a further embodiment, the structure carrying the bHMV is a micelle. A micelle is formed by the spontaneous organization of amphiphilic materials in solution into particles with a hydrophobic core and a hydrophilic corona. A micelle has little or no internal void. Therefore, micelles generally only carry bHMVs associated with the surface of the micelle or as part of the micellar structure. A micelle may also carry bHMV entrapped within the micellar structure.

bHMVs carried by micelles are preferably modified to be amphiphilic or are linked to a lipophilic anchor such that they are incorporated into the micellar structure. Likewise, a hydrophilic bHMV may be carried within a micelle's hydrophilic corona. Techniques for constructing micelles and for incorporating materials into micelles or onto micellar surfaces are well known in the art; see for example Torchilin V P, (1997) Q J Nuclear Med. 41:141-153; Weissig V et al., (1998) Pharm. Res. 15:1552-1556; Gabizon et al., P.N.A.S., USA, 18:6949-53 (1988), the disclosures of which are herein incorporated by reference in their entirety.

Micelles may also be modified to affect their biodistribution, for example by association with opsonization inhibitors or targeting groups. A surface modifier may be associated with a micelle by attachment (e.g. covalent or ionic bond, or other means of chemical or electrochemical linkage or interaction) to the micellar surface, or by incorporation of the surface modifier into the micellar structure. For example, a long-chain hydrophilic polymer or a targeting group may be conjugated to a lipophilic anchor and assembled into the micellar structure along with the other amphiphilic material. Examples of opsonization inhibiting moieties and targeting groups useful for modifying micelles are as described for liposomes above. Techniques for modifying micelles are well known in the art, see e.g. Torchilin V P, (1997) Q J Nuclear Med. 41:141-153; Weissig V et al., (1998) Pharm. Res. 15:1552-1556; Gabizon et al., P.N.A.S., USA, 18:6949-53 (1988).

Micelles are particularly useful in delivering bHMVs to tumors residing in the lymphatic system, especially when administered by subcutaneous injection or infusion. Micelles modified with opsonization inhibiting moieties are useful in delivering bHMVs to solid tumors when administered intravascularly, as micelles will accumulate in tissue fed by porous or leaky microvasculature (see Gabizon et al., supra).

A preparation of micelles typically has a distribution of sizes. A micelle preparation a size distribution within less than about a 20% standard deviation of the average diameter is preferred. A micelle preparation in a with a size distribution within about 10% of the average diameter is still more preferred. Preferably, the micelles of the invention are between about 5 nanometers and about 50 nanometers in diameter.

Pharmaceutical formulations of micelles may be prepared as described above for liposomes, using techniques well known to those of skill in the art.

In a still further embodiment, the structure carrying a bHMV is a microcapsule. Microcapsules are fine dispersions of solids or droplets of liquid onto which a thin film coating has been applied. The average diameter of microcapsules may vary from one micron to several hundred microns depending on the materials used and their method of production. The coating of microcapsules comprises an non-amphiphilic organic polymer, including for example amines (e.g. mono-, di-, tri-, tetra-, and higher amines, mixtures thereof, and mixtures thereof with monoamines), alginic acid, arabic acid, cellulose sulfate, carboxymethylcellulose, carrageenans, chondroitin sulfate, heparin, polyacrylic acid, polyoxyethylene cross-linked polyacrylic acid, polyphosphazine, glycolic acid esters of polyphosphazine, lactic acid esters of polyphosphazine, hyaluronic acid, polygalacturonic acid, polyphenylene sulfonic acid, and polyvinylcarboxylic acid, polymerizable aldehydes, derivatives thereof and mixtures thereof. Examples of microcapsules suitable for use in the present invention are found in U.S. Pat. No. 5,686,113 of Speaker et at., U.S. Pat. No. 5,501,863 of Rossling et al. and U.S. Pat. No. 5,993,374 of Kick, the disclosures of which are incorporated herein by reference in their entirety.

Microcapsules may optionally be modified to affect their biodistribution, for example to alter the microcapsule biodistribution by addition of targeting groups or opsonization-inhibiting moieties. Microcapsules modified in this way have similar characteristics and advantages as modified liposomes and micelles. Opsonization inhibiting moieties and targeting groups useful in surface-modifying microcapsules are as described for liposomes and micelles above. Techniques for preparing and modifying microcapsules are well known in the art (see, for example, U.S. Pat. Nos. 5,686,113, 5,501,863, and 5,993,374, supra).

The microcapsules of the invention may be any size. Particularly preferred microcapsules are those which are small enough to pass through the pulmonary capillary bed; i.e. those with an diameter of approximately 8 microns. However, microcapsules useful in the present invention may have a diameter of about 0.1 to about 2,000 microns. Preferred microcapsules are those having a diameter of about 100 to 1,000 microns, others having a diameter of about 10 to 100 microns, and still others having a diameter of about 1 to 100 microns. A preparation of microcapsules typically has a distribution of sizes. A microcapsule preparation in a range of about 0.1 to 10 microns with a size distribution within less than about a 20% standard deviation of the average diameter is preferred. A microcapsule preparation in a range of about 0.1 to 10 microns with a size distribution within about 10% of the average diameter is still more preferred.

Pharmaceutical formulations of microcapsules may be prepared as described above for liposomes and micelles, using techniques well known to those of skill in the art (see, for example, U.S. Pat. No. 5,501,863, supra).

The types of mutations that can be corrected by methods of the present invention are termed “vector-repairable mutations.” Vector-repairable mutations are mutations other than a large insertions, large deletions, or intrachromosomal rearrangements. A large insertion or deletion mutation is a mutation in which more than five contiguous nucleotides are inserted or deleted compared to the normal or wild type sequence. In particular, vector-repairable mutations include, for example, any mutation caused by the replacement of a nucleotide by a different nucleotide, including replacements of up to 5 contiguous nucleotides. Vector-repairable mutations also include, for example, any mutation caused by the insertion or deletion of up to 5, preferably up to 3, contiguous nucleotides. A vector-repairable mutation may reside in any portion of a human gene, e.g., exons, introns or other intervening sequences, or in a control element, e.g., a promoter or enhancer. The invention also encompasses the treatment of diseases caused by several vector-repairable mutations in the same gene. In this case, the disease may be treated by introducing mixtures of multiple bHMVs into the cells of the subject. Each bHMV is constructed to repair one of the vector-repairable mutations present within the gene.

The present invention thus encompasses a method of treating a genetic disease with a bHMV containing a sequence which can repair a mutated gene. Non-limiting examples of genetic diseases that may be treated by the repair of vector-repairable mutations found in hematopoietic cells include, for example:

hemoglobinopathies caused by mutations in a globin structural gene, such as sickle cell disease;

diseases caused by a mutation in the beta-globin gene such as beta-thalassemia; and

forms of Gaucher Disease that are caused by one or more mutations in the glucocerebrosidase structural gene.

These diseases are described in U.S. Pat. Nos. 5,760,012 and 5,888,983 of Kmiec et al., the entire disclosures of which are incorporated herein by reference. Examples of genetic diseases that may be treated by repair of vector-repairable mutations in hepatocytes include, for example:

familial hypercholesteremia, caused by mutation in the LDL receptor;

emphysema that caused by a mutation in the alphal -antitrypsin gene; and

hemophilia and Christmas Disease, which are caused by mutations in coagulation factors VIII and IX, respectively.

If not well known, a vector-repairable mutation may be characterized by techniques known to those of ordinary skill in the art. Preferably, the target gene from the subject to be treated is sequenced to identify the location and nature of the vector-repairable mutation. For example, more than 500 point mutations in globin genes have been described. (Bunn, H. F. & Forget, B. G., 1986, Hemoglobin: Molecular, Genetic and Clinical Aspects, W. B. Saunders, Philadelphia, PA). Similarly, no one mutation causes Gaucher Disease (Hong, C. M., et al., 1990, DNA Cell Biol. 9:233-41) or beta-thalassemia (Kazazian, H. H., 1990, Seminars in Hematology 27:209-228).

In another embodiment of the invention, bHMVs may be used to construct transgenic non-human organisms. A transgenic non-human organism constructed using a bHMV may be an animal; for example a mammal, bird, fish or reptile. The transgenic non-human organism constructed using a bHMV may also be a plant.

In one embodiment, transgenic non-human organisms may be constructed by introducing one or more bHMVs into gametes or zygotes derived from syngamic eukaryotic organisms, such that one or more target sequences in the gametes or zygotes are altered. The alteration in the target sequence of the gamete or zygote effected by a bHMV becomes part of the genotype of the organism that develops from the altered gamete or zygote. The genetic alteration passed on by the altered gamete or zygote to some or all cells of the mature transgenic organism preferably also affects the organism's phenotype.

Syngamic organisms include, for example, amphibians, reptiles, birds, mammnals, bony fishes, cartilaginous fishes, cyclostomes, arthropods, insects, mollusks, thallophytes, and embryophytes including gymnospermns and angiosperms. Preferred syngamic organisms include mammals, birds, fishes, gymnosperms and angiosperms. The present method is particularly useful in constructing transgenic non-human mammals; for example rodents (e.g. mice, rats and guinea pigs), rabbits, ovine mammals (e.g. sheep and goats), bovine mammals (e.g. cows), and porcine mammals (e.g. pigs). The present method is also useful in constructing transgenic plants, especially plants of agricultural value including, but not limited to: corn, tobacco, tomato, potato, soybean, and wheat.

The bHMV may be introduced directly into the nucleus of a gamete, e.g., a mature sperm, egg or polar body, for example by microinjection. A gamete transfected with a bHMV is an “altered gamete.” A second gamete or other suitable cell is then fused with the altered gamete to form an altered zygote. An altered zygote may also be created by direct injection of a bHMV into a zygote.

The particular type of gamete for bHMV introduction is not critical to zygote formation. It is not critical that the bHMV be introduced into any one type of gamete prior to zygote formation. For example, either a sperm cell or an egg may be injected with a bHMV and then fused with a complementary gamete. Alternatively, a polar body may be injected with a bHMV and fused with an egg, or vice versa. If the bHMV is introduced into a mature sperm cell prior to fertilization of an egg with that sperm cell, the sperm cell is preferably induced to undergo decondensation of its chromatin. Otherwise, the chromosomal complement of the sperm cell may be too dense to allow the addition of any material to its nucleus. Techniques for decondensation of sperm cells are known in the art. See for example Mahi, D. A., et al, J. Reprod. Fert., 44:293-296 (1975); Hendricks, D. M. et al, Exptl. Cell Res., 40:402-412 (1965); and Wagner, T. E., et al, Archives of Andrology, 1:31-41 (1978), the entire disclosures of which are incorporated herein by reference. Decondensation of sperm cells through the use of a disulfide reductant is preferred. The sperm cell into which the bHMV is introduced may thereafter be fused with an ovum to enable the formation of a zygote.

In a preferred embodiment, the bHMV is introduced into either the male or female pronucleus of a zygote. See Brinster, RL et al. (1989) PNAS USA 86:7087 and U.S. Pat. No. 4,873,191 of Wagner and Hoppe, the entire disclosures of which are incorporated herein by reference. It is desirable to introduce the bHMV into either the male or the female pronucleus as soon as possible after the sperm enters the egg; i.e., immediately after the formation of the male pronucleus when the pronuclei are clearly defined, well separated, and located near the zygote plasma membrane. The male pronucleus is the preferred site for introduction of the bHMV. Techniques for obtaining germ cells from syngamic organisms, especially mammals and plants, are well known in the art.

An altered zygote is preferably implanted into a foster mother so that the altered zygote may develop into a mature organism. For example, multiple zygotes may be altered with bHMVs and implanted into one or more foster mothers, and the foster mothers allowed to carry the implanted altered zygotes to term. Techniques for implantation of zygotes into foster mothers are well known in the art. If the altered zygote is implanted in a foster mother, it is preferred that implantation occurs at the morula or blastocyst stage of development. An altered zygote may be maintained in culture until the morula or blastocyst stage is reached.

In another embodiment, a nonhuman transgenic animal may be constructed by introducing a bHMV into an embryonic stem (ES) cell derived from an animal. ES cells may be obtained from, for example, mammals, birds, fishes, or reptiles. This technique is particularly useful in constructing transgenic mammals; for example primates (excluding humans), rodents (e.g. mice, rats and guinea pigs), rabbits, ovine mammals (e.g. sheep and goats), bovine mammals (e.g. cows), and porcine mammals (e.g. pigs). Methods of obtaining embryonic stem cells and normal blastocyst cells are known in the art; see for example Thomson et al., (1998) Science 282:1145-47 and Reubinoff et al. (2000) Nat. Biotechnol. 18:399-404 (both incorporated herein by reference). A bHMV introduced into ES cells produces ES cells with an altered target gene sequence. Chimeric animals are then produced by aggregation of altered ES cells with normal blastocyst cells. Transgenic animals are recovered as offspring of the chimeric animals, according to the method of Capecchi, M. R., 1989, Science 244:1288, the entire disclosure of which is incorporated herein by reference.

In particular, embryonic stem cells may be treated with a bHMV designed to introduce a frameshift or stop codon into a particular gene to produce a “knockout” transgenic animal. A “knockout” transgenic animal is a transgenic animal which has lost the function of one or more genes. Preferably, multiple alternative bHMVs, each encoding redundant stop codons, may be introduced into the embryonic stem cells.

The bHMVs may also be used to construct transgenic plants, especially within the dicotyledonous species. Transgenic plants include plants (as well as parts and cells of said plants) and their progeny, which have had target gene sequences altered by one or more bHMVs. The bHMV-induced alteration can, for example, cause a change in expression of a particular gene, such as to enhance or suppress production of a particular gene. For example, the growth characteristics of a plant can be altered by manipulating the expression of endogenous genes (see Carango et al., Biochem. Biophys. Res. Commun. (1988)152(3):1348-52; Shah, DM et al. (1986) Science 233:478; Comai L et al. (1985) Nature 317:741; and Hichee etal. (1988) Bio/Technology 6:915), or the color and/or pattern of flower petals can be changed by the induction or suppression of endogenous genes. Increased resistance to insects, pesticides and herbicides may also be conferred by a bHMV-induced genetic alteration.

Any method for direct DNA introduction in protoplasts and plant cells or tissues may be used to deliver the bHMV. Such methods include, for example, microinjection, electroporation, particle bombardment and direct oligonucleobase uptake; see Gasser CS and Fraley RT (1989) Science 244:1293 and Potrykus, I. (1990) Bio/Technol. 8, 535, the entire disclosures of which are incorporated herein by reference. For example Potrykus, supra, describes the transformation of protoplasts using the calcium/polyethylene glycol method. U.S. Pat. No. 6,013,863 of Lundquist, et al. discloses the production of transgenic corn plants by particle bombardment (incorporated herein by reference). Preferred methods for introducing bHMVs into monocotyledonous plants include microprojectile bombardment of explants or suspensions of cells, and direct DNA uptake or electroporation of protoplasts. Methods for obtaining mature plants from transfected cells, protoplasts or tissues are well known in the art; see for example EP 449,375 and U.S. Pat. No. 6,013,863 (supra).

Thus, the invention provides a method of constructing transgenic plants wherein one or more bHMV's designed to alter a target gene sequence is introduced into a plant cell or protoplast, and a mature plant is obtained from the transfected plant cell or protoplast.

Within the context of the present invention, plants to be selected include, but are not limited to, crops producing edible flowers such as cauliflower (Brassica oleracea), artichoke (Cynara scolymus), fruits such as apple (Malus, e.g. domesticus), banana (Musa, e.g. acuminata), berries (such as the currant, Ribes, e.g. rubrum), cherries (such as the sweet cherry, Prunus, e.g. avium), cucumber (Cucumis, e.g. sativus), grape (Vitis, e.g. vinifera), lemon (Citrus limon), melon (Cucumis melo), nuts (such as the walnut, Juglans, e.g. regia; peanut, Arachis hypogeae), orange (Citrus, e.g. maxima), peach (Prunus, e.g. persica), pear (Pyra, e.g. communis), plum (Prunus, e.g. domestica), strawberry (Fragaria, e.g. moschata), tomato (Lycopersicon, e.g. esculentum), leafs, such as alfalfa (Medicago, e.g. sativa), cabbages (e.g. Brassica oleracea), endive (Cichoreum, e.g. endivia), leek (Allium, e.g. porrum), lettuce (Lactuca, e.g. sativa), spinach (Spinacia e.g. oleraceae), tobacco (Nicotiana, e.g. tabacum), roots, such as arrowroot (Maranta, e.g. arundinacea), beet (Beta, e.g. vulgaris), carrot (Daucus, e.g. carota), cassava (Manihot, e.g. esculenta), turnip (Brassica, e.g. rapa), radish (Raphanus, e.g. sativus), yam (Dioscorea, e.g. esculenta), sweet potato (Ipomoea batatas) and seeds, such as bean (Phaseolus, e.g. vulgaris), pea (Pisum, e.g. sativum), soybean (Glycin, e.g. max), wheat (Triticum, e.g. aestivum), barley (Hordeum, e.g. vulgare), corn (Zea, e.g. mays), rice (Oryza, e.g. sativa), rapeseed (Brassica napus), millet (Panicum L.), sunflower (Helianthus annus), oats (Avena sativa), tubers, such as kohlrabi (Brassica, e.g. oleraceae), potato (Solanum, e.g. tuberosum) and the like, and ornamental flowering plants such as roses (including Gallicas, Albas, Damasks, Damask Perpetuals, Centifolias, Chinas, Teas and Hybrid Teas) and ornamental goldenrods (e.g. Solidago spp.).

For any transgenic organism, the presence of a target gene alteration introduced by a bHMV may be confirmed by, for example, sequence analysis or by PCR amplification using primers specific for the altered gene. For a “knockout” transgenic organism, loss of a functional gene may be confirmed by functional assay for the gene product.

bHMVs may also be used to mutagenize a population of cells to produce mutant cells having a selectable phenotype. Thus the invention provides a method of selecting the mutated cells from among a larger number of unmutated cells. According to one embodiment, a mixture of bHMVs having a constant targeting strand and different mutator strands is provided. For example, the mismatch region of each mutator strand may comprise different combinations of three non-wild type nucleotides. Introducing the bHMV mixture into a population of target cells induces a variety of mutations in the target cells. The mutations may fortuitously confer a selectable phenotype on certain cells within the population. Selectable phenotypes include, for example, growth advantages such as drug resistance, alterations in growth regulation and an altered capacity to use metabolites. Mutants having a selectable phenotype may be isolated under appropriate culture conditions.

The selection method may comprise negative selection. Negative selection is a technique whereby the mutated cells are rendered incapable of growth under the selecting conditions. The unmutated cells are removed by exposure to conditions which selectively destroy proliferating cells. Alternatively, the mutated cells may have an altered cell-surface antigenic phenotype that can be detected by immunofluorescence. Selection can be performed, for example by, fluorescence activated cell sorting (FACS).

The practice of the invention requires introduction the bHMV into a cell, in particular a eukaryotic cell. The bHMV may be introduced directly into the cell nucleus or pronucleus, or may be introduced into the cell cytoplasm. Any method for the introduction of genetic material into prokaryotic or eukaryotic cells may be used. The process of introducing genetic material into a target cell is termed “transfection”.

Transfection methods for prokaryotic cells are well known in the art, and are outlined, for example, in Molecular Cloning, a Laboratory Manual. Transfection methods for eukaryotic cells are also well known in the art, and include, for example, direct injection into the nucleus or pronucleus, electroporation, liposome transfer and calcium phosphate precipitation. In a preferred method, the transfection is performed with a liposomal transfer compound, e.g., DOTAP (N-[1-(2,3-dioleoyloxy)propyl]-N,N,N-trimethylammonium methylsulfate, Boehringer-Mannheim) or an equivalent, such as LIPOFECTIN. The amount of bHMV used is not critical to the practice of the invention; acceptable results may be achieved with 10 mM/10⁵ cells. A ratio of about 500 nanograms of bHMV in 3 micrograms of DOTAP per 10⁵ cells may be used.

A particularly preferred transfection method is electroporation. Using electroporation, as many as 1 cell per 10,000 treated cells can be specifically mutated at the target sequence (hereinafter “transformed”). Typically, useful amounts of a chimeric vector are between 10 and 1000 ng of chimeric vector per million cultured cells to be transformed by electroporation.

When the method of introducing the bHMV into the cell is direct injection, as for example when constructing transgenic animals by pronuclear injection, the rate of transformation can be greater than 1 per 10,000 cells, and the need for selection is thereby considerably reduced.

The invention will now be illustrated by the following non-limiting examples.

### EXAMPLE 1

Preparation of a bHMV targeting the kanamycin gene

Two oligonucleotides, targeting strand A and mutator strand B, were synthesized and purified using standard techniques. Both targeting strand A and mutator strand B folded into a hairpin containing a six base pair stem and a four base loop. A 26 base-long, single-stranded overhang extended from the 5′ end of the hairpin in each strand.

The overhang of targeting strand A comprised the targeting strand homologous region and contained a chimeric backbone in which five DNA residues were flanked by ten 2′-O-methyl RNA residues. The targeting strand homologous region of targeting strand A was homologous to a portion of the mutant kanamycin resistance gene (the target gene sequence). The overhang of mutator strand B comprised the mutator strand complementary region, and contained a one-base mismatch to the targeting strand homologous region of targeting strand A (and thus was also a mismatch to the target gene sequence). This mismatch was designed to correct a point mutation in the kanamycin resistance gene. The 3′ ends of targeting strand A and mutator strand B were blocked with a propylamine group. The primary sequences of targeting strand A and mutator strand B are given below.

Targeting Strand A:

| | |
|---|---|
| 5′-gu-ugu-gcc-cag-TCC-TAg-ccg-aau-agc-CTC-TCC-TTT-TGG-AGA-G-3′ | (SEQ ID NO:4) |

Mutator Strand B:

| | |
|---|---|
| 5′-GCT-ATT-CGG-CTA-CGA-CTG-GGC-ACA-ACA-GAC-AAT-TTT-TTG-TC-3′ | (SEQ ID NO:5) |

Lowercase letters represent 2′-O-methyl RNA, uppercase letters represent DNA, and the underlined base in mutator strand B denotes the mismatched base.

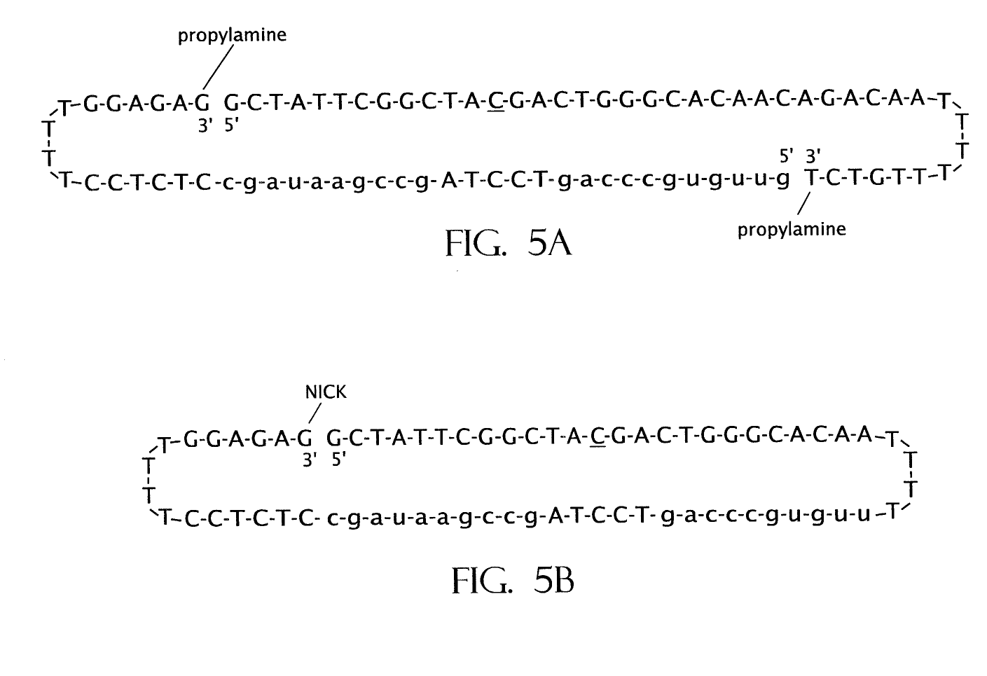

Targeting strand A and mutator strand B were mixed in equal amounts in water, the solution heated at 90° C. for 2 minutes and 60° C. for 10 minutes, and then cooled to room temperature for 30 minutes to form bHMV A/B. Formation of bHMV A/B was verified by electrophoresis in a 5% 3:1 NuSieve agarose gel (FMC Corp.), as bHMV A/B electrophoresed slower than either targeting strand A or mutator strand B. A schematic of bHMV A/B is shown in FIG. 5A.

### EXAMPLE 2

Correction of a mutant kanamycin gene in a human cell-free extract with bHMV A/B as compared to duplex mutational vector S

bHMV A/B from Example 1 was tested for gene repair activity as compared to the analogous singly-nicked duplex mutational vector S. The primary sequence of duplex mutational vector S is shown below, and a schematic of this vector is given in FIG. 5B.

Primary Sequence of Duplex Mutational Vector S

| | |
|---|---|
| 5′-GCT-ATT-CGG-CTA-CGA-CTG-GGC-ACA-ATT-TTu-ugu-gcc-cag-TCC- | (SEQ ID NO:6) |
| TAg-ccg-aau-agc-CTC-TCC-TTT-TGG-AGA-G-3′ | |

As above, the lowercase letters represent 2′-O-methyl RNA, the uppercase letters represent DNA, and the underlined base denotes the mismatched base.

Duplex mutational vector S differs from bHMV A/B in that bHMV A/B contains two nicks (i.e. comprises two oligonucleotide strands) and duplex mutational vector S contains one nick (i.e., comprises one oligonucleotide strand). bHMV A/B also has an additional 7 base-pairs relative to duplex mutational vector S (see FIG. 5A, where the additional base pairs of bHMV A/B are shown in bold). It is not expected that these additional base pairs contributed significantly to the enhancement of gene repair frequency exhibited by bHMV A/B as compared to duplex mutation vector S; see Gamper et al., (2000) “A plausible mechanism for gene correction by chimeric oligonucleotides,” Biochemistry, in press, who report that single nick duplex mutational vectors in which the length of homology is increased from 24 to 29 bases, the frequency of gene repair was enhanced by about 25%. As will be shown below, the increase in gene repair achieved with bHMVs as compared to single nick vectors is approximately 100%.

bHMV A/B and duplex mutational vector S were assayed for their ability to repair the mutant kanamycin gene in a human cell-free system using the protocol described in Cole-Strauss et al., Nucleic Acids Research (1999) 27, 1323-1330 (incorporated herein by reference). The mutant kanamycin gene was provided on an exogenous plasmid called the “kan⁻ plasmid.”

Briefly, cell-free extract was prepared from HuH-7 cells and split into two experimental samples and one control sample. bHMV A/B and the kan⁻ plasmid were added to the first experimental sample, and duplex mutational vector S and the kan⁻ plasmid were added to the second experimental sample. The control sample received only the kan⁻ plasmid. All samples were incubated for 45 minutes at 37° C., and then extracted twice with phenol/chloroform (1:1) and precipitated with ethanol to recover the kan- plasmid. The kan- plasmid recovered from each sample was dissolved in water, and an aliquot electroporated into competent E. coli DH10B cells. Serial dilutions of the electroporated DH10B cells from each sample were plated onto solid media containing kanamycin or ampicillin. Colonies were counted the following day. Ampicillin resistance served as a marker for successful transfection of the kan⁻ plasmid into the DH10B cells, since the plasmid also carried a wild-type ampicillin resistance gene. Kanamycin resistance indicated the mutant kanamycin gene on the kan⁻ plasmid had been repaired. The ratio of kan+/amp+ colonies reflected the fraction of kan⁻ plasmid in a particular sample that had undergone successful repair of the mutant kanamycin sequence. No repair was seen in the control sample. In multiple experiments, bHMV A/B exhibited an approximately 100% increase in the repair of the mutant kanamycin gene as compared to duplex mutational vector S.

### EXAMPLE 3

Correction of a mutant kanamycin gene in cultured human cells.

Hela cells were grown in DMEM at 37° C. and 5% CO₂ in a humidified incubator to a density of 2×10⁵ cells/ml in an 8 chamber slide (Lab-Tek). After replacing the regular DMEM with an alpha-MEM based culture medium called OptiMEM (Gibco/BRL No. 31885-013), cells were co-transfected with 10 micrograms of kan- plasmid from Example 2 that had been mixed with 5 micrograms of either bHMV A/B (see Example 1) or duplex mutational vector S (see Example 2). Each plasmid/vector mix had previously been complexed with 10 micrograms Lipofectamine (Life Technologies) according to the manufacturer's directions. The cells were treated with the lipofectamine-plasmid-vector mix for 6 hours at 37° C. Treated cells were washed with PBS and fresh DMEM was added. After a 16-18 hour recovery period, the cells was assayed for gene repair. Correction of the point mutation in the mutant kanamycin gene eliminated a stop codon and restored full expression. Kanamycin expression was detected by adding a small non-fluorescent ligand which bound a C—C—X—X—C—C sequence in the genetically modified carboxy terminus of the kanamycin protein to produce a highly fluorescent complex (FlAsH system, Aurora Biosciences Corporation). Following a 60 minute incubation at room temperature with the ligand (FIAsH-EDT2), cells expressing full length kanamycin product acquired an intense green fluorescence detectable by fluorescence microscopy using a fluorescein filter set. In Hela cells, bHMV A/B was approximately twice as effective as duplex mutational vector S in correcting the mutant kanamycin gene.

### EXAMPLE 4

Construction of Transgenic Mice by Direct Injection of bHMV into the Male Pronuclei of Fertilized Eggs

Fertilized eggs (i.e., zygotes) are collected at the early pronuclear stage from the oviducts of C57BL/6J female mice which have been mated to LT/Sv males. The male and female pronuclei are separate and distinguishable within the cytoplasm of the zygotes. The cumulus cells surrounding the zygotes are removed in culture medium containing 1 mg/ml bovine testis hyaluronidase, as described in Biol. Reprod., 8:420-426 (1973). Zygotes from several females are then pooled and are washed in fresh culture medium. The zygotes are maintained in a depression slide containing culture medium overlayered with paraffin oil in an atmosphere of 5% carbon dioxide, 5% oxygen and 90% nitrogen at 37° C. A bHMV is prepared according to Example 1 above.

Injection pipets with an external diameter of about 1 micrometer and holding pipets with an external diameter of about 60-70 micrometers are prepared from Pyrex tubing as described in Proc. Natl. Sci. U.S.A., 74:5657-5661 (1977), the entire disclosure of which is incorporated herein by reference. Manipulation of the pipets for holding and subsequent injection of the zygote is accomplished using Leitz micromanipulators and paraffin oil-filled Hamilton microsyringes. A small drop of culture medium containing 5-6 zygotes, and a small drop of a 10 mM bHMV in water are separately placed on a microscope slide and each drop is covered with paraffin oil. Approximately 10 picoliters of the bHMV solution is drawn into the injection pipet, which is then positioned over the culture medium drop containing the zygotes. Within the culture medium drop, a zygote is positioned onto the holding pipet so that the male pronucleus can be easily injected with the bHMV solution in the injection pipet. The tip of the injection pipet is inserted into the male pronucleus and the bHMV solution injected. This injection process is repeated for all of zygotes within the culture medium drop. The injected zygotes are then removed from the culture medium drop and placed in culture tubes where they are allowed to develop for five days. The conditions of the preimplantation development are as in Biol. Reprod., 8:420-426 (1973), the entire disclosure of which is incorporated herein by reference. Zygotes developing to morulae or blastocyst stage are transplanted into uteri of Fl hybrid C57BL/6J X SJL/J foster mouse mothers who are at day three of pseudopregnancy. The foster mothers carry the implanted morulae or blastocysts to term. Transgenic progeny are confirmed by PCR amplification of altered regions in the genomic DNA.

### EXAMPLE 5

Production of Fertile Transgenic Zea mays Plants with Resistance to Glyphosate

I. Initiation and Maintenance of Maize Cell Cultures which Retain Plant Regeneration Capacity

Friable, embryogenic maize callus cultures are initiated from hybrid immature embryos produced by pollination of inbred line A188 plants (University of Minnesota, Crop Improvement Association) with pollen of inbred line B73 plants (Iowa State University). Ears are harvested when the embryos reach a length of 1.5 to 2.0 mm. The whole ear is surface-sterilized in 50% v/v commercial bleach (2.63% w/v sodium hypochlorite) for 20 min. at room temperature. The ears are then washed with sterile distilled, deionized water. Immature embryos are aseptically isolated and placed on nutrient agar initiation/maintenance media with the root/shoot axis exposed to the medium. The initiation/maintenance media, hereinafter referred to as F medium, consists of N6 basal media (Chu et al., 1975, Sci Sin (Peking) 18:659-668) with 2% (w/v) sucrose, 1.5 milligrams per liter 2,4-dichlorophenoxyacetic acid (2,4-D), 6 mM proline, and 0.25% Gelrite (Kelco, Inc.,of the F-medium San Diego). The pH is adjusted to 5.8 prior to autoclaving. Unless otherwise stated, all tissue culture manipulations are carried out under sterile conditions.

The immature embryos are incubated at 26° C. in the dark. Cell proliferations from the scutellum of the immature embryos are evaluated for friable consistency and the presence of well defined somatic embryos. Tissue with this morphology is transferred to fresh media 10 to 14 days after the initial plating of the immature embryos. The tissue is then subcultured on a routine basis every 14 to 21 days. Sixty to eighty milligram quantities of tissue are removed from pieces of tissue which have reached a size of approximately one gram, and are transferred to fresh media for subculturing. Subculturing requires careful visual monitoring to ensure that only tissue of the correct morphology is maintained. The presence of somatic embryos ensures that the cultures give rise to plants under the proper conditions.

II. Binary Hybrid Mutational Vectors

A transfection mixture of bHMVs targeting the maize 5-enolpyruvylshikimate-3-phosphate synthase (ESPS) gene (see H. Steinrucken and N. Amrhein, (1980) Biochem. Biophys. Res. Commun. 94:1207 and DM Mousedale and J R Coggins, (1984) Planta 160:78, both incorporated herein by reference) is provided. Each bHMV in the transfection mixture contains a constant targeting strand and a different mutator strand. The mismatch region of each mutator strand consists of a different random combination of three non-wild type nucleotides. Introducing the bHMV transfection mixture into a population of target eukaryotic cells induces a variety of mutations in the target cells. The set of mutations introduced by the transfection mixture may fortuitously confer an increased glyphosate resistance in certain cells of the Zea mays cultures. Glyphosate is a common agricultural herbicide (e.g., sold as Roundup™ brand herbicides, manufactured by Monsanto Co.). Glyphosate resistance may be conferred by mutations causing either overproduction of normal ESPS or glyphosate-tolerant ESPS.

III. bHMV Delivery Process

The bHMV transfection mixture is introduced into the embryogenic maize callus cultures prepared above by bombardment with microparticles coated with the transfection mixture. Bombardment of the cultured calli is accomplished with a “gene-gun” bombardment instrument obtained from Biolistics, Inc. (Ithaca, N.Y.). Microparticles, macroprojectiles and stopping plates are obtained from Biolistics, Inc., and are sterilized as described by the supplier.

The embryogenic maize callus cultures are subcultured 7 to 12 days prior to bombardment, and are prepared for bombardment as follows: Sample plate are made by arranging five clumps of callus, each approximately 50 mg in wet weight, in a cross pattern in the center of a sterile 60×15 mm petri plate (Falcon 1007). Sample plates are stored in a closed container with moist paper towels throughout the bombardment process. Ten sample plates are prepared.

The bHMV transfection mixture is coated onto M-10 tungsten particles (Biolistics) essentially as described by Klein et al. (1988b), the entire disclosure of which is incorporated herein by reference, as follows: Two Eppendorf microcentrifuge tubes are prepared which contain 25 microliters of 50 mg/ml M-10 tungsten particles in water, 25 microliters 2.5 M CaCl₂, and 10 microliters 100 mM spermidine free base. Five microliters of the bHMV transfection ok- mixture (5 nanograms/microliter total vector concentration) are added to each tube. Both tubes are incubated on ice for 10 minutes to allow the bHMV mixture to coat the tungsten particles. The coated particles are then pelleted in an Eppendorf centrifuge at room temperature for 5 seconds. Twenty five microliters of the supernatant is discarded, leaving 65 microliters of supernatant overlaying a pellet of coated tungsten particles. The tubes are stored on ice throughout the bombardment process. Each tube is used for five bombardments.

For bombardment, each sample plate tray is positioned 5 centimeters below the bottom of the stopping plate tray of the microprojectile instrument. The stopping plate is set in the slot below the barrel. The sample plate is centered on the sample plate tray and the sample plate's lid is removed. A 7×7 centimeter square rigid wire frame supporting a 3×3 millimeter galvanized steel mesh is placed over the open sample dish in order to retain the tissue during the bombardment. Each tube containing the coated particle preparations described above is sonicated as described by Biolistics, Inc., and 2.5 microliters of the resuspended coated particle preparation is pipetted onto the top of macroprojectiles supplied by Biolistics, Inc. Bombardment is carried out as described by the manufacturer. Each sample plate is bombarded once.

IV. Selection Process

Immediately after all samples plates have been bombarded, five 30 milligram pieces of callus per sample plate are transferred, plate for plate, onto selection plates containing glyphosate in F medium. Glyphosate is incorporated into the F medium by addition of the appropriate volume of filter sterilized glyphosate when the F-medium cools to 45° C. prior to pouring the selection plates. Only callus tissue containing cells successfully transfected with the bHMV transfection mixture, and which have acquired a mutation or mutations conferring glyphosate resistance from the transfection mixture, will proliferate on the selection plates. After two weeks of selection, portions of callus tissue proliferating on the selection plates (i.e., exhibiting glyphosate resistance) are transferred to growth plates containing F medium without glyphosate.

V. Characterization of Mutations in Transfected Calli

Genomic DNA is isolated from portions of glyphosate resistant callus tissue obtained from the growth plates. Mutations imparted to the ESPS gene in each tissue sample are characterized by PCR amplification of genomic ESPS sequences, using primers corresponding to the ESPS gene sequence flanking the mutator region mismatches of each bHMV from the transfection mixture. Genomic DNA is isolated from the callus tissue according to the procedure of Lundquist et al., U.S. Pat. No. 6,013,863. The PCR reactions are performed, and the PCR products isolated and sequenced, by techniques known to those skilled in the art.

VI. Plant Regeneration and Production of Seed

Callus tissue from the growth plates for which the ESPS mutations have been characterized are transferred to plates containing RM5 medium. RM5 medium consists of MS basal salts (Murashige, T, et al. (1962) Physiol Plant 15:473-497) supplemented with thiamine HCl 0.5 mg/l, 2,4-D 0.75 mg/Il, sucrose 50 g/l, asparagine 150 mg/l, and Gelrite 2.5 g/l (Kelco Inc. San Diego). After 14 days on RM5 medium, the callus tissue samples are transferred to R5 medium. R5 medium is identical to RM5 medium, except that 2,4-D is omitted. The callus tissue samples are cultured in the dark for 7 days at 26° C., and transferred to a light regime of 14 hours light and 10 hours dark for 14 days at 26° C. to allow plantlets to grow. Plantlets are then transferred to one quart canning jars (Ball) containing 100 ml of R5 medium, where they develop into plants after 14 to 21 days. Plants are transferred from the canning jars to vermiculite, and grown for 7 to 8 days before being transplanted into soil and grown to maturity. Glyphosate resistance in mature plants is shown by treatment of the plants with Roundup Ultra™ herbicide (Monsanto Co.).

VII. Sexual Reproduction of Glyphosate Resistant Plants

Controlled pollinations of mature plants are conducted by standard techniques with inbred lines A188, B73 and Oh43. Seed is harvested 45 days post-pollination and allowed to dry further 1-2 weeks. The harvested seeds are germinated, and progeny plants grown in the presence of Roundup Ultra™ herbicide. Progeny plants exhibit glyphosate resistance.

### EXAMPLE 6

In Vivo Targeted Repair of the Canine Dystrophin Gene

Duchenne muscular dystrophy (DMD) is an X-linked recessive neuromuscular disorder of humans, characterized by progressive muscle weakness and wasting accompanied by infiltration of fibrotic tissue into the skeletal muscle. Symptoms of the disease appear at about 2-4 years of age, and affected individuals usually die from cardiac or respiratory arrest in their late teens. A milder form of DMD, called Becker's muscular dystrophy (BMD), has the same symptoms but a slower clinical progression. BMD patients typically succumb to the disease between 20-30 years of age.

DMD is caused by point mutations, duplications or deletions in the dystrophin gene that cause a shift in the reading frame, resulting in a missing or non-functional dystrophin protein. The alleleic disease, Becker muscular dystrophy (BMD), is caused by deletions, duplications or missense mutations in the dystrophin gene which maintain the reading frame.

The role of dystrophin in the skeletal muscle fiber is not completely understood, but it is believed the protein forms part of the cell membrane scaffold that imports strength and resiliency to the muscle fiber membrane. Dystrophin has been localized to the inside surface of the muscle fiber membrane, and is believed to be associated with a number of other proteins found on and in the muscle fiber membrane.

Golden retriever muscular dystrophy (GRMD) is caused by a point mutation (A to G transition) in the splice acceptor site of intron 6 in the dystrophin gene of a certain strain of golden retriever dogs. This condition is analogous to DMD in humans. Functional dystrophin is not produced in detectable quantities in GRMD dogs, as the mutation causes the complete elimination of exon 7 from the mature mRNA and consequent shift in the reading frame. Correction of this point mutation alleviates the splicing defect and results in detectable dystrophin levels in the skeletal muscle fibers of treated GRMD animals. See Bartlett R J et al. (2000), Nat. Biotech. 18:615-622.

A bHMV, called “Exon 7 bHMV,” which corrects the GRMD defect in dystrophic dogs was designed, synthesized by standard techniques and purified by high pressure liquid chromatography (HPLC). Exon 7 bHMV was introduced into the skeletal muscle of an affected dog, and resulted in the expression of dystrophin protein containing exon 7, which localized to the cell membrane. The experiment was performed as follows:

The mutator strand of Exon 7 bHMV was a 44-nucleobase oligomer with the following sequence (SEQ ID NO:7). All nucleobases in the mutator strand were deoxyribonucleotides except for the four uracils (shown in lowercase), which are O-methylated ribonucleotide bases. The four O-methyl uracils form a hairpin structure which allows intra-strand base pairing between the six deoxyribonucleotides immediately 3′ of the hairpin with the six deoxyribonucleotides immediately 5′ of the hairpin. The mutator region of the bHMV comprised a five-base sequence (shown in bold) that is the complement of the wild-type coding strand sequence at the splice acceptor site of canine dystrophin intron 6, as reported in Bartlett R J et al., supra, FIG. 1A, the disclosure of which is herein incorporated by reference. The one-base mismatch, which should cause a reversion of the mutant G to the wild-type A in the canine dystrophin gene, is underlined.

| | |
|---|---|
| 5′-ATGTGTGTGTTTCAGGCCAGACCTGTTTGATTGGuuuuCCAATC-3′ | (SEQ ID NO:7) |

The targeting strand of Exon 7 bHMV was a 44-nucleobase oligomer with the following sequence (SEQ ID NO:8). All nucleobases were O-methylated ribonucleotide bases, except for the 5′-most nucleobase, which was a deoxyribonucleotide base. The O-methylated RNA bases are shown in lower case, and the deoxyribonucleotide base is shown in upper case. The four uracils toward the 3′ end (shown in italics) form a hairpin structure which allows intra-strand base pairing between the six deoxyribonucleotides immediately 3′ of the hairpin with the six deoxyribonucleotides immediately 5′ of the hairpin.

5′-Aaacaggucuggcccgaaacacacacaugcgcgcuuuugcgcgc-3′ (SEQ ID NO: 8)

The mutator and targeting strands were annealed to produce the Exon 7 bHMV by mixing equimolar quantities of each strand, heating the mixture to 78° C. for 15 minutes, cooling to room temperature for 10 minutes, and placing the mixture on ice. There was no ligation between the 5′ and 3′ ends of the strands.

350 micrograms of the Exon 7 bHMV was introduced into the cranial sartorius muscle in one leg of a 6 week old male GRMD dog by “square wave” electroporation (Cytopulse, Baltimore, Md.). As a control, the cranial sartorius muscle of the contralateral leg was sham-treated by electroporation with no bHMV.

Biopsies of the treated and control muscle were taken at 2 weeks and 2 months post-treatment. The biopsies were wrapped in aluminum foil, flash frozen in liquid nitrogen, and cut in cross-section with respect to the muscle fibers to a thickness of 6-8 microns, as described in Bartlett R J et al., supra. The biopsy sections were used for histochemical analysis, and for extraction of RNA (for RT/PCR of dystrophin mRNA), protein (for Western blots detection of dystrophin), and DNA (for PCR and sequencing of dystrophin sequences).

Dystrophin was detected in the biopsy sections by fluorescent immunohistochemistry with monoclonal antibodies specific for exon 7 or exon 8 of the normal canine dystrophin protein, as described in Bartlett R J et al., supra. At both time points, sham treated muscle showed no dystrophin staining, but the treated muscle showed intense staining for dystrophin at the fiber peripheries. These results indicate a stable conversion of the GRMD mutation to wild-type by the bHMV, with consequent production of functional dystrophin protein, in the treated muscle fibers.

### EXAMPLE 7

In Vivo Restoration of Reading Frame in Exon 8 of the Canine Dystrophin Gene

A second bHMV, called “Exon 8 bHMV,” was designed, synthesized by standard techniques, and purified by HPLC. The Exon 8 bHMV corrects the frameshift in exon 8 of the GRMD dystrophin gene.

The targeting strand of Exon 8 bHMV was a 49-nucleobase oligomer with the following sequence (SEQ ID NO:9). All nucleobases in the mutator strand were deoxyribonucleotides except for the four uracils (shown in lowercase), which are O-methylated ribonucleotide bases. The four O-methyl uracils form a hairpin structure which allows intra-strand base pairing between the six deoxyribonucleotides immediately 3′ of the hairpin with the six deoxyribonucleotides immediately 5′ of the hairpin.

| | |
|---|---|
| 5′-GGATAAGTGGTGGCAAATCTGTAAGCACATTAACACCAGuuuuCTGGTG-3′ | (SEQ ID NO:9) |

The targeting strand of Exon 8 bHMV was a 54-nucleobase oligomer with the following sequence (SEQ ID NO:10). All nucleobases were O-methylated ribonucleotide bases, except for the 5′-most nucleobase, which was a deoxyribonucleotide base. The O-methylated RNA bases are shown in lower case, and the deoxyribonucleotide base is shown in upper case. The four uracils toward the 3′ end (shown in italics) form a hairpin structure which allows intra-strand base pairing between the eight O-methylated ribonucleotides immediately 3′ of the hairpin with the eight O-methylated ribonucleotides immediately 5′ of the hairpin. The 3′ end of the targeting strand was blocked with an amine group to prevent ligation of the 3′ end to the 5′ end of the mutator strand.

| | |
|---|---|
| 5′-Tuaaugugcuuaagauguugccaccacuuauccgcgcgcgcuuuugcgcgcgc-3′ | (SEQ ID NO:10) |

The mutator and targeting strands were annealed to produce the Exon 8 bHMV by mixing equimolar quantities of each strand, heating the mixture to 78° C. for 15 minutes, cooling to room temperature for 10 minutes, and placing the mixture on ice. There was no ligation between the 5′ and 3′ ends of the strands. 350 micrograms of the Exon 7 bHMV was introduced into the cranial sartorius muscle in one leg of a 6 week old male GRMD dog by “square wave” electroporation (Cytopulse, Baltimore, Md.). As a control, the cranial sartorius muscle of the contralateral leg was sham-treated by electroporation with no bHMV.

All citations referred to herein are incorporated by reference. One skilled in the art would readily appreciate that the present invention is well adapted to carry out the stated objects and obtain the disclosed ends and advantages, as well as those inherent herein. The present invention may be embodied in other specific forms without departing from the spirit or essential attributes thereof. Therefore, any descriptions of specific embodiments in the foregoing disclosure is not to be construed as limiting the scope of the present invention.

We claim:

1. A binary hybrid mutational vector comprising an oligonucleobase targeting strand and an oligonucleobase mutator strand, wherein said targeting strand comprises:
a) a 3′ end and a 5′ end; and
b) a targeting strand homologous region comprising a sequence of nucleobases homologous to a target gene sequence;
and wherein said mutator strand comprises:
c) a 3′ end and a 5′ end;
d) a complementary region comprising a sequence of nucleobases complementary to the targeting strand homologous region; and
e) a mismatch region located within the complementary region.

2. The binary hybrid mutational vector of claim 1, wherein said targeting strand homologous region comprises at least 70% ribo-type nucleobases, and said complementary region and said mismatch region comprise at least 70% deoxyribo-type nucleobases.

3. The binary hybrid mutational vector of claim 1, wherein said targeting strand comprises at least 70% ribo-type nucleobases and said mutator strand comprises at least 70% deoxyribo-type nucleobases.

4. The binary hybrid mutational vector of claim 2, wherein at least one ribo-type nucleobase is nuclease resistant.

5. The binary hybrid mutational vector of claim 4, wherein said nuclease-resistant ribo-type nucleobase is selected from the group consisting of 2′AX-nucleosides, 2′AX-nucleotoids and 2′AR-nucleotides, wherein:
A is oxygen or a halogen;
X is hydrogen or C₁₋₆ alkyl; and
R is C₁₋₆ alkyl;
provided when A is a halogen, then X and R are omitted.

6. The binary hybrid mutational vector of claim 5, wherein said halogen is fluorine, chlorine or bromine.

7. The binary hybrid mutational vector of claim 2, wherein said ribo-type nucleobases are ribonucleotides, and said deoxyribo-type nucleobases are deoxyribonucleotides.

8. The binary hybrid mutational vector of claim 7, wherein at least one ribonucleotide is nuclease resistant.

9. The binary hybrid mutational vector of claim 7, wherein said nuclease-resistant ribonucleotide is a 2′AR-nucleotide, wherein:
A is oxygen or a halogen; and
R is C₁₋₆ alkyl;
provided when A is a halogen, then R is omitted.

10. The binary hybrid mutational vector of claim 9, wherein said nuclease-resistant ribonucleotide is a 2′-O-methyl ribonucleotide.

11. The binary hybrid mutational vector of claim 3, wherein at least one ribo-type nucleobase is nuclease resistant.

12. The binary hybrid mutational vector of claim 11, wherein said nuclease-resistant ribo-type nucleobase is selected from the group consisting of 2′AX-nucleosides, 2′AX-nucleotoids and 2′AR-nucleotides, wherein
A is oxygen or a halogen;
X is hydrogen or C₁₋₆ alky; and
R is C₁₋₆ alkyl;
provided when A is a halogen, then X and R are omitted.

13. The binary hybrid mutational vector of claim 12, wherein said halogen is fluorine, chlorine or bromine.

14. The binary hybrid mutational vector of claim 3, wherein said ribo-type nucleobases are ribonucleotides, and said deoxyribo-type nucleobases are deoxyribonucleotides.

15. The binary hybrid mutational vector of claim 14, wherein at least one ribonucleotide is nuclease resistant.

16. The binary hybrid mutational vector of claim 15, wherein said nuclease-resistant ribonucleotide is a 2′AR-nucleotide, wherein:
A is oxygen or a halogen; and
R is C₁₋₆ alkyl;
provided when A is a halogen, then R is omitted.

17. The binary hybrid mutational vector of claim 16, wherein said nuclease-resistant ribonucleotide is a 2′-O-methyl ribonucleotide.

18. The binary hybrid mutational vector of claim 1, wherein:
(a) said targeting strand further comprises a 3′ hairpin formed by an intra-strand duplex region of base pairs linked by a region of contiguous unpaired bases, and the targeting strand homologous region is located adjacent to the 3′ hairpin and extends in the 5′ direction along the targeting strand; and
(b) said mutator strand farther comprises a 3′ hairpin formed by an intra-strand duplex region of base pairs linked by a region of contiguous unpaired bases, and the complementary region is located adjacent to the 3′ hairpin and extends in the 5′ direction along the mutator strand.

19. The binary hybrid mutational vector of claim 18 wherein the nucleobases are nucleotides.

20. The binary hybrid mutational vector of claim 19 wherein:
(a) said targeting strand further comprises a 3′-terminal ribonucleotide which is blocked from ligation and the 3′ hairpin is formed by an intra-strand duplex region of 4-8 ribonucleotide base pairs linked by a region of four contiguous unpaired uracil ribonucleotides; and
(b) said mutator strand further comprises a 3′-terminal ribonucleotide which is blocked from ligation and the 3′ hairpin is formed by an intra-strand duplex region of 4-8 deoxyribonucleotide base pairs linked by a region of four contiguous unpaired thymine deoxyribonucleotides.

21. The binary hybrid mutational vector of claim 20 wherein at least one ribonucleotide is nuclease resistant.

22. The binary hybrid mutational vector of claim 21 wherein said nuclease-resistant ribonucleotide is a 2′AR-nucleotides, wherein:
A is oxygen or a halogen; and
R is C₁₋₆ alkyl;
provided when A is a halogen, then R is omitted.

23. The binary hybrid mutational vector of claim 22, wherein said ribonucleotides are 2′-O-methyl ribonucleotides.

24. The binary hybrid mutational vector of claim 1, wherein:
a) the mutator strand is longer than the targeting strand, and further comprises a hairpin at each end of the oligonucleobase chain having a stretch of single-stranded nucleobases disposed therebetween, wherein the stretch of single-stranded nucleobases comprises the complementary region; and
b) the targeting strand is substantially single-stranded and the targeting strand homologous region is coextensive with the targeting strand.

25. The binary hybrid mutational vector of claim 24, wherein said targeting strand comprises at least 70% ribo-type nucleobases and said mutator strand comprises at least 70% deoxyribo-type nucleobases.

26. The binary hybrid mutational vector of claim 25, wherein said ribo-type nucleobases are ribonucleotides, and said deoxyribo-type nucleobases are deoxyribonucleotides.

27. The binary hybrid mutational vector of claim 26, wherein at least one ribonucleotide is nuclease resistant.

28. The binary hybrid mutational vector of claim 27, wherein said nuclease-resistant ribonucleotide is a 2′AR-nucleotide, wherein:
A is oxygen or a halogen; and
R is C₁₋₆ alkyl;
provided when A is a halogen, then R is omitted.

29. The binary hybrid mutational vector of claim 28, wherein said nuclease-resistant ribonucleotide is a 2′-O-methyl ribonucleotide.

30. A method of producing a binary hybrid mutational vector comprising the steps of:
(a) providing a separately synthesized oligonucleobase targeting strand and oligonucleobase mutator strand, wherein said targeting strand comprises:
1) a 3′ end and a 5′ end; and
2) a targeting strand homologous region comprising a sequence of nucleobases homologous to a target gene sequence;
and wherein said mutator strand comprises:
3) a 3′ end and a 5′ end;
4) a complementary region comprising a sequence of nucleobases complementary to the targeting strand homologous region; and
5) a mismatch region located within the complementary region; and
b) mixing said targeting strand and said mutator strand such that the mutator strand complementary region containing the mismatch region forms a duplex with the targeting strand homologous region.

31. The method of claim 30, wherein said targeting strand homologous region comprises at least 70% ribo-type nucleobases, and said mutator strand complementary and mismatch regions comprise at least 70% deoxyribo-type nucleobases.

32. The method of claim 30, wherein said targeting strand comprises at least 70% ribo-type nucleobases and said mutator strand comprises at least 70% deoxyribo-type nucleobases.

33. The method of claim 31, wherein said ribo-type nucleobases are ribonucleotides, and said deoxyribo-type nucleobases are deoxyribonucleotides.

34. The method of claim 32, wherein said ribo-type nucleobases are ribonucleotides, and said deoxyribo-type nucleobases are deoxyribonucleotides.

35. The method of claim 33 or 34, wherein at least one ribonucleotide is a 2′-O-methyl ribonucleotide.

36. An oligonucleobase set comprising an oligonucleobase targeting strand and a plurality of oligonucleobase mutator strands, wherein said targeting strand comprises:
a) a 3′ end and a 5′ end; and
b) a targeting strand homologous region comprising a sequence of nucleobases homologous to a target gene sequence;
and wherein each mutator strand comprises:
c) a 3′ end and a 5′ end;
dd) a complementary region comprising a sequence of nucleobases complementary to the targeting strand homologous region; and
e) a unique mismatch region located within the complementary region.

37. The oligonucleobase set of claim 36, wherein the targeting strand is hybridized to each mutator strand to form an array of binary hybrid mutational vectors.

38. The oligonucleobase set of claim 36 wherein the targeting strand and each mutator strand are provided in separate containers.

39. A kit comprising:
1) the oligonucleobase set of claim 37; and
2) instructions for introducing said binary hybrid mutational vectors into cells.

40. A kit comprising:
1) the oligonucleobase set of claim 38;
2) instructions for hybridizing said targeting and mutator strands to produce a binary hybrid mutational vector; and
3) instructions for introducing said binary hybrid mutational vector into a cell.

41. A method of altering a target gene sequence, comprising the steps of:
a) providing a cell having a target gene sequence; and
b) introducing the binary hybrid mutational vector of claim 1 into said cell, so that said binary hybrid mutational vector alters said target gene sequence.

42. The method of claim 41, wherein said targeting strand comprises at least 70% ribo-type nucleobases and said mutator strand comprises at least 70% deoxyribo-type nucleobases.

43. The method of claim 42, wherein said ribo-type nucleobases are ribonucleotides and said deoxyribo-type nucleobases are deoxyribonucleotides.

44. The method of claim 43, wherein said ribonucleotides are 2′-O-methyl ribonucleotides.

45. The method of claim 41 wherein said cell is a eukaryotic cell.

46. The method of claim 45 wherein said cell is a mammalian cell.

47. The method of claim 46 wherein said mammalian cell is an ovum with a pronucleus, and said binary hybrid mutational vector is introduced by direct injection into said pronucleus.

48. The method of claim 46 wherein said mammalian cell is an embryonic stem cell.

49. The method of claim 45 wherein said eukaryotic cell is a plant cell.

50. A method of treating a genetic disease, comprising:
a) providing a subject having at least one vector-repairable mutation associated with said disease;
b) placing a population of cells from said subject in culture, wherein said cells carry the mutation;
c) introducing a binary hybrid mutational vector or mixture of binary hybrid mutational vectors of claim 1 designed to correct said vector-repairable mutation into said cells to form mutation-corrected cells; and
d) reimplanting said mutation-corrected cells into the subject.

51. The method of claim 50 wherein the subject is a human.

52. The method of claim 50, wherein said targeting strand comprises at least 70% ribo-type nucleobases and said mutator strand comprises at least 70% deoxyribo-type nucleobases.

53. The method of claim 52, wherein said ribo-type nucleobases are ribonucleotides and said deoxyribo-type nucleobases are deoxyribonucleotides.

54. The method of claim 53, wherein said ribonucleotides are 2′-O-methyl ribonucleotides.

55. The method of claim 50 wherein said cells are selected from the group consisting of hematopoietic cells, hematopoietic stem cells, hepatocytes, hepatic reserve cells and embryonic stem cells.

56. The method of claim 50 wherein said genetic disease is selected from the group consisting of hemoglobinopathies, beta-thalassemia, Gaucher Disease, familial hypercholesterolemia, emphysema, hemophilia and Christmas Disease.

57. A method of selecting mutated cells from among unmutated cells comprising the steps of:
a) providing a population of cells to be mutagenized;
b) transfecting said population of cells with at least one binary hybrid mutational vector of claim 1 to produce mutant cells having a selectable phenotype; and
c) isolating said mutants having the selectable phenotype with appropriate culture conditions.

58. A method of constructing a transgenic mammal, comprising:
a) obtaining a mammalian gamete;
b) providing one or more binary hybrid mutational vectors of claim 1 for alteration of a target gene sequence in the gamete;
c) introducing said one or more binary hybrid mutational vectors into the nucleus of the gamete to produce an altered gamete and fusing the altered gamete with another cell to form an altered zygote;
d) implanting the altered zygote into a female mammal capable of bearing the altered zygote to term; and
e) allowing the zygote to develop to term.

59. A method of constructing a transgenic mammal, comprising:
a) obtaining a mammalian zygote;
b) providing one or more binary hybrid mutational vectors of claim 1 for alteration of a target gene sequence in the zygote;
c) introducing said one or more binary hybrid mutational vectors into the nucleus or pronucleus of the zygote to form an altered zygote;
d) implanting the altered zygote into a female capable of bearing the zygote to term; and
e) allowing the zygote to develop to term.

60. A method of constructing a transgenic plant, comprising:
a) providing a plant cell or protoplast containing a target gene sequence;
b) introducing the binary hybrid mutational vector of claim 1 into said plant cell or protoplast, so that said binary hybrid mutational vector alters said target gene sequence to form an altered plant cell or an altered protoplast; and
c) obtaining a mature plant from the altered plant cell or the altered protoplast.

61. The method of claim 60 wherein said binary hybrid mutational vector is introduced by microinjection, electroporation, particle bombardment or direct oligonucleobase uptake.

62. The binary hybrid mutational vector of claim 1, wherein the binary hybrid mutational vector is modified for in vivo administration.

63. The binary hybrid mutational vector of claim 1 wherein the binary hybrid mutational vector is carried by a structure.

64. The binary hybrid mutational vector of claim 63 wherein said structure is selected from the group consisting of liposomes, micelles and microcapsules.

65. The binary hybrid mutational vector of claim 64 wherein said structure is modified to affect its biodistribution.

66. A sterile, pyrogen-free pharmaceutical composition comprising the binary hybrid mutational vector of claim 62 or 65.

67. A method of altering a target sequence on a target cell in vivo, comprising the steps of:
a) providing a subject having a target cell with a target gene sequence; and
b) administering the binary hybrid mutational vector of claim 1 into said subject in vivo, so that said binary hybrid mutational vector alters the target gene sequence in the target cell.

68. The method of claim 67 wherein the binary hybrid mutational vector is modified for in vivo administration.

69. The method of claim 67 wherein the binary hybrid mutational vector is carried by a structure.

70. The method of claim 69 wherein said structure is selected from the group consisting of liposomes, micelles and microcapsules.

71. The method of claim 70 wherein said structure is modified to affect its biodistribution.
